(19) United States
(12) Patent Application Publication
(10) Pub. No.: US 2023/0019690 A1
(43) Pub. Date: Jan. 19, 2023

(54) FORMULATIONS COMPRISING A MINERAL AND/OR A VITAMIN AND A POLYSACCHARIDE, COMPOSITIONS THEREOF AND USE THEREOF IN SUPPLEMENTING SAID MINERAL AND/OR VITAMIN

(71) Applicant: ALESCO S.R.L., Pisa (PI) (IT)

(72) Inventors: Andrea LACORTE, PISA (IT); Germano TARANTINO, PISA (IT); Elisa BRILLI, PISA (IT)

(21) Appl. No.: 17/782,140

(22) PCT Filed: Dec. 4, 2020
(86) PCT No.: PCT/IB2020/061521

§ 371 Date: Jun. 2, 2022

(30) Foreign Application Priority Data
Dec. 4, 2019 (IT) 102019000023016

(51) Int. Cl. A61K 31/714; A61K 33/06; A61K 33/30; A61K 33/26; A61K 33/18; A61K 33/04; A61K 33/34; A61K 33/24; A61K 31/375; A61K 31/593; A61K 31/355; A61K 47/24; A61K 47/36

(52) U.S. Cl. CPC A61K 31/714; A61K 33/06; A61K 33/30; A61K 33/26; A61K 33/18; A61K 33/04; A61K 33/34; A61K 33/24; A61K 31/375; A61K 31/593; A61K 31/355; A61K 47/24; A61K 47/36

(57) ABSTRACT

A solid form formulation based on a nutrient comprising: (a) a mineral or a vitamin, (b) a phospholipid, (c) a first agent selected from (c-i) carrageenan and (c-ii) acacia gum, and, optionally, (d) a sucrester and/or (e) a starch of plant origin and related composition, process of preparation and method of treatment of a deficiency of said mineral and/or vitamin are disclosed.

The present invention relates to a solid form formulation based on a nutrient (such as a mineral and/or a vitamin) comprising or, alternatively, consisting of: (a) at least one mineral and/or at least one vitamin, (b) at least one phospholipid, (c) at least one polysaccharide and, optionally, (d) at least one sucrester and/or (e) at least one starch of plant origin.

In a first embodiment of said formulation, the present invention relates to a solid form formulation based on a mineral (referred to as cyclosome or cyclosomal mineral) comprising or, alternatively, consisting of: (a) at least one mineral in the form of a salt or complex or oxide of said mineral, such as magnesium, calcium, iron, zinc, iodine, selenium, chromium and/or copper, (b) at least one phospholipid, (c) at least one first agent (polysaccharide) selected from (c-i) at least one carrageenan, (c-ii) at least one acacia gum, (c-iii) at least one fucoidan and mixtures thereof, and, optionally, said formulation further comprises (d) at least one sucrester and/or (e) at least one starch of plant origin.

In a second embodiment of said formulation, the present invention relates to a solid form formulation based on a vitamin (referred to as cyclosome or cyclosomal vitamin) comprising or, alternatively, consisting of: (a) at least one vitamin, such as a vitamin A, a vitamins of group B (preferably B12), a vitamin C, a vitamin D (preferably D3) and/or a vitamin E, (b) at least one phospholipid, (c) at least one first agent (polysaccharide) selected from (c-i) at least one carrageenan, (c-ii) at least one acacia gum (c-iii) at least one fucoidan and mixtures thereof, and, optionally, said formulation further comprises (d) at least one sucrester and/or (e) at least one starch of plant origin.

In a third embodiment of said formulation, the present invention relates to a solid form formulation based on at least one mineral and at least one vitamin (referred to as, cyclosome or cyclosomal mineral-vitamin) comprising or, alternatively, consisting of: (a) at least one mineral and at least one vitamin, as defined above, (b) at least one phospholipid, (c) at least one first agent selected from (c-i) at least one carrageenan, (c-ii) at least one acacia gum, (c-iii) at least one fucoidan and mixtures thereof, and, optionally, said formulation further comprises (d) at least one sucrester and/or (e) at least one starch of plant origin.

Furthermore, the present invention relates to a composition, preferably in solid form, comprising at least one additive and at least one of said solid form formulations comprising at least one nutrient (i.e. at least one mineral and/or at least one vitamin) and to the use of said compositions or formulations in the treatment (therapeutic or non-therapeutic) of a deficiency of said at least one nutrient, and of diseases, symptoms and/or disorders related to or deriving from said deficiency.

In a first embodiment of said composition, the present invention relates to a composition, preferably in solid form, comprising additives and said solid form formulation comprising at least one mineral (cyclosomal mineral) and to the use of said composition or formulation in the treatment (therapeutic or non-therapeutic) of a deficiency of said at least one mineral, and of diseases, symptoms or disorders related to or deriving from said deficiency.

In a second embodiment of said composition, the present invention relates to a composition, preferably in solid form, comprising additives and said solid form formulation comprising at least one vitamin (cyclosomal vitamin) and to the use of said composition or formulation in the treatment (therapeutic or non-therapeutic) of a deficiency of said at least one vitamin, and of diseases, symptoms or disorders related to or deriving from said deficiency.

In a third embodiment of said composition, the present invention relates to a composition, preferably in solid form, comprising additives and at least one of said solid form formulation comprising a mineral (cyclosomal mineral) and at least one of said solid form formulation comprising a vitamin (cyclosomal vitamin) and to the use of said composition in the treatment (therapeutic or non-therapeutic) of a deficiency of said mineral and/or vitamin, and of diseases, symptoms or disorders related to or deriving from said deficiency.

In a fourth embodiment of said composition, the present invention relates to a composition, preferably in solid form, comprising additives and said solid form formulation comprising at least one mineral and at least one vitamin (cyclosomal mineral-vitamin) and to the use of said composition or formulation in the treatment (therapeutic or non-therapeutic) of a deficiency of said at least one mineral and at least one vitamin, and of diseases, symptoms or disorders related to or deriving from said deficiency.

Lastly, the present invention relates to a process for the preparation of said solid form formulation comprising said at least one mineral (cyclosome or cyclosomal mineral) or said solid form formulation comprising a vitamin (cyclosome or cyclosomal vitamin) or of said solid form formulation comprising at least one mineral and at least one vitamin (cyclosome or cyclosomal mineral-vitamin).

In addition, the present invention relates to a further process for the preparation of said composition comprising at least one formulation comprising a mineral (cyclosomal mineral) and/or at least one formulation comprising a vitamin (cyclosomal vitamin) (in short, composition process of the invention).

Each of the minerals and/or vitamins which may be present in the compositions or formulations of the present invention (such as magnesium, calcium, iron, zinc, iodine, selenium, chromium and copper, or vitamin A, of group B, C, D and E), plays fundamental roles in the metabolism and functioning and homeostasis mechanisms of cells and organs in subjects, particularly in human subjects.

For example, iron is used to treat sideropenia, as well as anaemia and to increase haemoglobin and ferritin values in subjects; magnesium is used to treat musculoskeletal disorders, cardiometabolic disorders, emotional sphere disorders (e.g. stress) and immune disorders (e.g. physical and mental fatigue); calcium is used to treat disorders related to pregnancy (i.e. development of the foetus), mood disorders, bone disorders, muscle disorders and pressure disorders; zinc is used to treat growth and development disorders, metabolism disorders, immune system disorders, vision disorders and cognitive disorders; selenium is used to treat disorders related to pregnancy (i.e. development of the foetus), metabolic disorders and immune system disorders; iodine is used to treat disorders related to pregnancy (i.e. development of the foetus), mood disorders, pressure disorders, metabolism disorders, cardiovascular disorders and energy deficiency disorders; and chromium is used to treat changes in the carbohydrate, lipid and energy metabolism.

As a matter of fact, chromium is an essential element in cellular energy metabolism, both of carbohydrates and lipids, capable of enhancing the effects of insulin by lowering blood sugar and favouring the entry of amino acids and lipids into cells.

Lastly, copper is involved in redox reactions and protein synthesis, for example for the production of enzymes, in the human organism plays a fundamental role in the constitution of the biological respiratory catalyst cytochrome C oxidase.

However, when said minerals (such as magnesium, calcium, iron, zinc, iodine, selenium, copper and/or chromium) and/or said vitamins (such as vitamin A, of group B (e.g. B12), C, D (e.g. D3) and/or E) are administered through the oral route (in short, per os) to a subject, preferably a human subject, their gastrointestinal or intestinal absorption and its blood bioavailability may vary from subject to subject and/or not be particularly high and therefore cause significant differences in the response of subjects, with cases of poor efficacy.

Therefore, there is a high need to provide novel formulations of said minerals (such as magnesium, calcium, iron, zinc, iodine, selenium, copper and/or chromium) and/or of said vitamins (such as vitamin A, of group B (e.g. B12), C, D (e.g. D3) and/or E) and compositions comprising said formulations which are both highly absorbable at the gastrointestinal level and bioavailable at the blood level and highly effective in the supplementation of said mineral and/or vitamin, in the treatment of a insufficiency or deficiency of said mineral and/or vitamin and in the treatment of diseases, symptoms or disorders related to or deriving from such deficiency, for all categories of subjects.

For example, diseases, symptoms or disorders related to or deriving from an insufficiency or deficiency of said mineral, such as magnesium, calcium, iron, zinc, iodine, selenium, copper and/or chromium, can be selected from: changes in the carbohydrate metabolism and/or diseases and disorders related thereto, such as, for example, diabetes, hyperglycaemia, insulin resistance, high absorption of carbohydrates, deregulation of blood glucose level and/or metabolic syndrome; changes in the muscle energy metabolism and/or disorders related thereto, such as, for example, decrease in muscle mass, decrease in muscle strength, decrease in physical resistance to muscle stress, poor absorption of amino acids; dyslipidaemia or change in the lipid metabolism and/or diseases and disorders related thereto, such as, for example, cholesterolaemia, high triglyceride levels, and obesity or overweight; cognitive disorders; cardiometabolic disorders.

For example, diseases or symptoms related to or deriving from an insufficiency or deficiency of said vitamins (such as, vitamin A, of group B (e.g. B12), C, D (e.g. D3) and/or E) may be selected from: cognitive problems, motor problems, decrease in immune defences and others as exemplified in the present description.

Lastly, the need is felt for said novel solid form formulations of said at least one mineral and/or of a vitamin and the compositions thereof to also be stable over time, well tolerated by all categories of subjects and easy to prepare.

Following extensive research and development activity, the Applicant addresses and solves the aforementioned needs by providing novel solid form formulations comprising said at least one mineral (such as magnesium, calcium, iron, zinc, iodine, selenium, copper and/or chromium) and/or a vitamin (such as vitamin A, of group B (e.g. B12), C, D (e.g. D3) and/or E) and compositions, preferably in solid form, comprising additives and said formulations comprising said at least one mineral and/or at least one vitamin.

Said formulations based on at least one mineral and/or based on at least one vitamin of the present invention and the compositions thereof are effective in the treatment of a partial or almost total deficiency of said at least one mineral and/or of said at least one vitamin, and in the treatment of diseases, symptoms or disorders related to or deriving from said deficiency.

An object of the present invention is to provide a novel formulation of said at least one mineral (such as, magnesium, calcium, iron, zinc, iodine, selenium, copper and/or chromium) and/or novel formulation of at least one vitamin (such as, vitamin A, of group B (e.g. B12), C, D (e.g. D3) and/or E), and compositions thereof which are capable of making the supplementation of said mineral administered through the oral route effective, for example following an increased gastrointestinal absorption and blood bioavailability of the mineral.

In the context of the present invention, the terms gastrointestinal absorption and intestinal absorption are used interchangeably.

A good level of efficacy and/or increased efficacy, even by a small extent, of the formulation based on at least one mineral and/or of the formulation based on at least one vitamin according to the present invention with respect to the mineral as such (or a salt or complex or oxide thereof) and/or to the vitamin as such in the treatment of a deficiency of said mineral and/or of said vitamin, or in the treatment of diseases/disorders deriving from said deficiency and illustrated in the present invention, results in a decrease in the effective dose to be administered to a subject in need with respect to the mineral as such (or a salt or complex or oxide thereof) and/or to the vitamin as such, and it is therefore also cost-effective.

Said good level or increased efficacy, even by a slight extent, in the aforementioned treatments with minerals and/or vitamins formulated according to the invention or compositions thereof, with respect to the treatments with the mineral as such (or a salt or complex or oxide thereof) and/or with the vitamin as such, could be due to an increased blood bioavailability of the mineral and/or vitamin, in turn due to an increased intestinal or gastrointestinal absorption of the formulations based on the mineral and/or vitamin subject of the present invention with respect to the mineral as such (or a salt or complex or oxide thereof) and/or vitamin as such, when administered orally. However, said increased efficacy could be due to other mechanisms and reasons.

It is conceivable that the increased intestinal absorption of the formulations or compositions based on the mineral and/or vitamin subject of the present invention with respect to the mineral as such (or a salt or complex or oxide thereof) and/or vitamin as such, is due to the internalisation of said formulations in microvesicles which allow the transport thereof through the intestinal membrane.

Said good level or increased efficacy in the treatment of a deficiency of said mineral and/or of a deficiency of said vitamin or in the treatment of diseases/disorders deriving from said deficiency by using the formulations or compositions based on the mineral and/or based on a vitamin subject of the present the invention, with respect to the mineral as such (or a salt or complex or oxide thereof) and/or to the vitamin as such, results in attaining a greater effect with the same amount or concentration of mineral and/or vitamin administered to a subject in need.

In addition, the formulations and compositions comprising said at least one mineral and/or said at least one vitamin subject of the present invention, are stable over time from the chemical/physical and organoleptic point of view.

Lastly, the formulations and compositions comprising said at least one mineral and/or said at least one vitamin subject of the present invention do not show any relevant side effects, they are well tolerated and, therefore, can be administered, even on an empty stomach, to all categories of subjects, including subjects in paediatric age, adolescents, pregnant or breastfeeding women and the elderly.

Furthermore, the process for the preparation of said solid form formulations comprising said at least one mineral (such as, magnesium, calcium, iron, zinc, iodine, selenium, copper and/or chromium) and/or of solid form formulations comprising at least one vitamin (such as, vitamin A, of group B (e.g. B12), C, D (e.g. D3) and/or E) and the compositions thereof are easy to carry out or prepare and cost-effective in proportion to the treatment potential.

For example, the solid form formulation based on at least one mineral and/or at least one vitamin subject of the invention (cyclosome or cyclosomal mineral and/or vitamin) can be processed easily to provide the compositions of the present invention, preferably in solid form for oral use.

These and other objects which will be apparent from the detailed description that follows, are achieved by the solid form formulation of said at least one mineral (such as, magnesium, calcium, iron, zinc, iodine, selenium, copper and/or chromium) and/or by the solid form formulation of at least one vitamin (such as vitamin A, of group B (e.g. B12), C, D (e.g. D3) and/or E) of the present invention (cyclosome or cyclosomal mineral and/or vitamin) and the compositions thereof thanks to the technical characteristics claimed in the attached claims and reported in the present description.

### FIGURES

FIG. 1: chart representing the amount of Fe³⁺ passing through the intestinal epithelium over time (rat isolated model).

FIG. 2: chart representing the kinetics of release of Fe³⁺ from the compositions according to the invention or comparative in simulated gastric environment pH 1.2.

FIG. 3: histogram representing the dimensions (nm) of the vesicles formed by the compositions according to the invention or comparative.

In the context of the present invention, the term “mineral” is used to indicate a salt (cation and anion), oxide, complex, cation (in the biologically active oxidation state), anion or aggregate.

### SUMMARY OF THE INVENTION

An object of the present invention is to provide a solid form formulation of at least one mineral and/or a solid form formulation of at least one vitamin comprising, besides said mineral and/or vitamin, a phospholipid, a polysaccharide and, optionally, a sucrester and/or a plant starch (in short, formulation of the present invention).

An object of the present invention is to provide a composition, preferably in solid form, comprising at least one formulation of the present invention (based on a mineral and/or a vitamin) and pharmaceutical or food grade additives and/or excipients (in short, composition of the present invention).

An object of the present invention is to provide said formulations or compositions of the present invention for use as medicament.

An object of the present invention is to provide said formulations or compositions of the present invention for use in a method for the treatment of a deficiency of said at least one mineral and/or at least one vitamin, to a subject in need.

An object of the present invention is to provide a method for the treatment of a deficiency of said at least one mineral and/or at least one vitamin by administering a therapeutically effective amount of said formulations or compositions of the present invention to a subject in need.

An object of the present invention is to provide a non-therapeutic (or cosmetic) use of said formulations or compositions of the present invention for the supplementation of said at least one mineral and/or at least one vitamin to a healthy subject in order to increase the physical and/or mental performance thereof.

### DETAILED DESCRIPTION OF THE INVENTION

Forming an object of the present invention is a solid form formulation of at least one mineral (in short, formulation of at least one mineral of the invention or formulation of the invention or, alternatively, cyclosome or cyclosomal mineral) comprising or, alternatively, consisting of:

(a) at least one mineral in the form of a salt or complex or oxide of said at least one mineral, wherein said mineral is selected from the group comprising or, alternatively, consisting of: (a-i) magnesium (II), (a-ii) calcium (II), (a-iii) iron (III) or iron (II), (a-iv) zinc (II), (a-v) iodine (V), (a-vi) selenium (IV), (a-vii) chromium (III), (a-viii) copper (II) and mixtures thereof;

(b) at least one phospholipid, preferably a phosphoglyceride, more preferably a phosphatidylcholine or lecithin;

(c) at least one first agent selected from the group comprising or, alternatively, consisting of: (c-i) at least one carrageenan, (c-ii) at least one acacia gum, (c-iii) at least one fucoidan, and mixtures thereof;

and, optionally, (d) at least one fatty acid carbohydrate ester (alternatively referred to as sucrester) and/or

(e) at least one gelatinised or pre-gelatinised starch of plant origin, preferably rice starch.

An example of said solid form formulation of at least one mineral comprises or, alternatively, consists of:

(a) at least one mineral selected from: (a-i) magnesium (II), (a-ii) calcium (II), (a-iii) iron (III) or iron (II), (a-iv) zinc (II), (a-v) iodine (V), (a-vi) selenium (IV), (a-vii) chromium (Ill), or (a-viii) copper (II);

(b) a lecithin, preferably a sunflower, corn or soy lecithin (preferably sunflower (E322));

(c-i) a carrageenan (e.g. E407) or (c-ii) an acacia gum (e.g. E414);

and, optionally, (d) a sucrester (e.g. sucrester E473);

and, optionally, (e) a gelatinised or pre-gelatinised starch of plant origin, preferably pre-gelatinised rice starch.

An example of said solid form formulation of at least one mineral comprises or, alternatively, consists of:

(a) at least one mineral selected from: (a-i) magnesium (II), (a-ii) calcium (II), (a-iii) iron (III) or iron (II), (a-iv) zinc (II), (a-v) iodine (V), (a-vi) selenium (IV), (a-vii) chromium (Ill), or (a-viii) copper (II);

(b) a lecithin, preferably a sunflower, corn or soy lecithin (preferably sunflower (E322));

(c-i) a carrageenan (e.g. E407) or (c-ii) an acacia gum (e.g. E414);

(d) a sucrester (e.g. sucrester E473);

and, optionally, (e) a gelatinised or pre-gelatinised starch of plant origin, preferably pre-gelatinised rice starch.

An example of said solid form formulation of at least one mineral comprises or, alternatively, consists of:

(a) at least one mineral selected from: (a-i) magnesium (II), (a-ii) calcium (II), (a-iii) iron (III) or iron (II), (a-iv) zinc (II), (a-v) iodine (V), (a-vi) selenium (IV), (a-vii) chromium (Ill), or (a-viii) copper (II);

(b) a lecithin, preferably a sunflower, corn or soy lecithin (preferably sunflower (E322));

(c-i) a carrageenan (e.g. E407) or (c-ii) an acacia gum (e.g. E414);

(d) a sucrester (e.g. sucrester E473); and

(e) a gelatinised or pre-gelatinised starch of plant origin, preferably pre-gelatinised rice starch.

In the context of the present invention, the term “mineral” is preferably used to indicate a mineral consisting of a single chemical element, for example, magnesium, calcium, iron, zinc, iodine, selenium, copper or chromium.

In the context of the present invention, the term “mineral salt” is used to indicate the salt of a mineral wherein said mineral is, preferably, a mineral in the form of cation (cation of the mineral) consisting of a single chemical element, for example, magnesium, calcium, iron, zinc, iodine, selenium or chromium.

In the context of the present invention, the term “cation of the mineral” (or mineral cation) is used to indicate the chemical form of a mono or multivalent cation of a mineral wherein said mineral is, preferably, a mineral consisting of a single chemical element, for example, magnesium, calcium, iron, zinc, iodine, selenium, copper or chromium.

In the context of the present invention the terms “mineral” and “cation of the mineral” (or mineral cation) are used interchangeably.

The following embodiments (FRs) of the solid form formulation of magnesium (in short, Mg) of the invention (cyclosome or cyclosomal magnesium) are comprised in the present invention:
FRa-Mg: Mg, (b) and (c) such as (c-i) or (c-ii) or (c-iii); for example Mg, (b) and (c-i) or Mg, (b) and (c-ii) or Mg, (b) and (c-iii).
FRb-Mg: Mg, (b), (c) such as (c-i) or (c-ii) or (c-iii) and (d); for example Mg, (b), (c-i) and (d) or Mg, (b), (c-ii) and (d) or Mg, (b), (c-iii) and (d).
FRc-Mg: Mg, (b), (c) such as (c-i) or (c-ii) or (c-iii) and (e); for example Mg, (b), (c-i) and (e) or Mg, (b), (c-ii) and (e) or Mg, (b), (c-iii) and (e).
FRd-Mg: Mg, (b), (c) such as (c-i) or (c-ii) or (c-iii), (d) and (e); for example Mg, (b), (c-i), (d) and (e) or Mg, (b), (c-ii), (d) and (e) or Mg, (b), (c-iii), (d) and (e).

wherein the components referred to as (a), (b), (c-i), (c-ii), (c-iii), (d) and (e) are as defined in the present invention.

In an embodiment of the invention, said solid form formulation of magnesium (referred to as cyclosome or cyclosomal magnesium) comprises or, alternatively, consists of:

(a-i) magnesium, preferably magnesium (II), more preferably magnesium in the form of magnesium oxide or magnesium hydroxide;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c) at least one first agent selected from the group comprising or, alternatively, consisting of: (c-i) at least one carrageenan, preferably carrageenan E407, (c-ii) at least one acacia gum, preferably acacia gum E414, (c-iii) at least one fucoidan, and mixtures thereof;

and, optionally,

(d) at least one sucrester, preferably sucrester E473; and/or

(e) at least one gelatinised or pre-gelatinised starch of plant origin, preferably pre-gelatinised rice starch.

The following embodiments (FRs) of the solid form formulation of calcium (in short, Ca) of the invention (cyclosome or cyclosomal magnesium) are comprised in the present invention:
FRa-Ca: Ca, (b) and (c) such as (c-i) or (c-ii) or (c-iii); for example Ca, (b) and (c-i) or Ca, (b) and (c-ii) or Ca, (b) and (c-iii).
FRb-Ca: Ca, (b), (c) such as (c-i) or (c-ii) or (c-iii) and (d); for example Ca, (b), (c-i) and (d) or Ca, (b), (c-ii) and (d) or Ca, (b), (c-iii) and (d).
FRc-Ca: Ca, (b), (c) such as (c-i) or (c-ii) or (c-iii) and (e); for example Ca, (b), (c-i) and (e) or Ca, (b), (c-ii) and (e) or Ca, (b), (c-iii) and (e).
FRd-Ca: Ca, (b), (c) such as (c-i) or (c-ii) or (c-iii), (d) and (e); for example Ca, (b), (c-i), (d) and (e) or Ca, (b), (c-ii), (d) and (e) or Ca, (b), (c-iii), (d) and (e).

wherein the components referred to as (a), (b), (c-i), (c-ii), (c-iii), (d) and (e) are as defined in the present invention.

In an embodiment of the invention, said solid form formulation of calcium (referred to as cyclosome or cyclosomal calcium) comprises or, alternatively, consists of:

(a-ii) calcium, preferably calcium (II), more preferably calcium in the form of tricalcium phosphate, for example tricalcium phosphate E341;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c) at least one first agent selected from the group comprising or, alternatively, consisting of: (c-i) at least one carrageenan, preferably carrageenan E407, (c-ii) at least one acacia gum, preferably acacia gum E414, (c-iii) at least one fucoidan, and mixtures thereof;

and, optionally,

(d) at least one sucrester, preferably sucrester E473; and/or

(e) at least one gelatinised or pre-gelatinised starch of plant origin, preferably pre-gelatinised rice starch.

According to a formulation embodiment of the present invention, when said mineral is calcium, the formulation of the invention does not comprise an acacia gum.

The following embodiments (FRs) of the solid form formulation of iron (in short, Fe) of the invention (cyclosome or cyclosomal magnesium) are comprised in the present invention:
FRa-Fe: Fe, (b) and (c) such as (c-i) or (c-ii) or (c-iii); for example Fe, (b) and (c-i) or Fe, (b) and (c-ii) or Fe, (b) and (c-iii).
FRb-Fe: Fe, (b), (c) such as (c-i) or (c-ii) or (c-iii) and (d); for example Fe, (b), (c-i) and (d) or Fe, (b), (c-ii) and (d) or Fe, (b), (c-iii) and (d).
FRc-Fe: Fe, (b), (c) such as (c-i) or (c-ii) or (c-iii) and (e); for example Fe, (b), (c-i) and (e) or Fe, (b), (c-ii) and (e) or Fe, (b), (c-iii) and (e).
FRd-Fe: Fe, (b), (c) such as (c-i) or (c-ii) or (c-iii), (d) and (e); for example Fe, (b), (c-i), (d) and (e) or Fe, (b), (c-ii), (d) and (e) or Fe, (b), (c-iii), (d) and (e).

wherein the components referred to as (a), (b), (c-i), (c-ii), (c-iii), (d) and (e) are as defined in the present invention.

In an embodiment of the invention, said solid form formulation of iron (referred to as cyclosome or cyclosomal iron) comprises or, alternatively, consists of:

(a-iii) iron, preferably iron (III), more preferably iron in the form of iron pyrophosphate;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c) at least one first agent selected from the group comprising or, alternatively, consisting of: (c-i) at least one carrageenan, preferably carrageenan E407, (c-ii) at least one acacia gum, preferably acacia gum E414, (c-iii) at least one fucoidan, and mixtures thereof;

and, optionally,

(d) at least one sucrester, preferably sucrester E473; and/or

(e) at least one gelatinised or pre-gelatinised starch of plant origin, preferably pre-gelatinised rice starch.

The following embodiments (FRs) of the solid form formulation of zinc (in short, Zn) of the invention (cyclosome or cyclosomal magnesium) are comprised in the present invention:
FRa-Zn: Zn, (b) and (c) such as (c-i) or (c-ii) or (c-iii); for example Zn, (b) and (c-i) or Zn, (b) and (c-ii) or Zn, (b) and (c-iii).
FRb-Zn: Zn, (b), (c) such as (c-i) or (c-ii) or (c-iii) and (d); for example Zn, (b), (c-i) and (d) or Zn, (b), (c-ii) and (d) or Zn, (b), (c-iii) and (d).
FRc-Zn: Zn, (b), (c) such as (c-i) or (c-ii) or (c-iii) and (e); for example Zn, (b), (c-i) and (e) or Zn, (b), (c-ii) and (e) or Zn, (b), (c-iii) and (e).
FRd-Zn: Zn, (b), (c) such as (c-i) or (c-ii) or (c-iii), (d) and (e); for example Zn, (b), (c-i), (d) and (e) or Zn, (b), (c-ii), (d) and (e) or Zn, (b), (c-iii), (d) and (e).

wherein the components referred to as (a), (b), (c-i), (c-ii), (c-iii), (d) and (e) are as defined in the present invention.

In an embodiment of the invention, said solid form formulation of zinc (referred to as cyclosome or cyclosomal zinc) comprises or, alternatively, consists of:

(a-iv) zinc, preferably zinc (IV), more preferably zinc in the form of zinc oxide;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c) at least one first agent selected from the group comprising or, alternatively, consisting of: (c-i) at least one carrageenan, preferably carrageenan E407, (c-ii) at least one acacia gum, preferably acacia gum E414, (c-iii) at least one fucoidan, and mixtures thereof;

and, optionally, (d) at least one sucrester, preferably sucrester E473; and/or

(e) at least one gelatinised or pre-gelatinised starch of plant origin, preferably pre-gelatinised rice starch.

The following embodiments (FRs) of the solid form formulation of iodine (in short, I) of the invention (cyclosome or cyclosomal magnesium) are comprised in the present invention:
FRa-I: I, (b) and (c) such as (c-i) or (c-ii) or (c-iii); for example I, (b) and (c-i) or I, (b) and (c-ii) or I, (b) and (c-iii).
FRb-I: I, (b), (c) such as (c-i) or (c-ii) or (c-iii) and (d); for example I, (b), (c-i) and (d) or I, (b), (c-ii) and (d) or I, (b), (c-iii) and (d).
FRc-I: I, (b), (c) such as (c-i) or (c-ii) or (c-iii) and (e); for example I, (b), (c-i) and (e) or I, (b), (c-ii) and (e) or I, (b), (c-iii) and (e).
FRd-I: I, (b), (c) such as (c-i) or (c-ii) or (c-iii), (d) and (e); for example I, (b), (c-i), (d) and (e) or I, (b), (c-ii), (d) and (e) or I, (b), (c-iii), (d) and (e).

wherein the components referred to as (a), (b), (c-i), (c-ii), (c-iii), (d) and (e) are as defined in the present invention.

In an embodiment of the invention, said solid form formulation of iodine (referred to as cyclosome or cyclosomal iodine) comprises or, alternatively, consists of:

(a-v) iodine, preferably iodine (V), more preferably iodine in the form of sodium iodate;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c) at least one first agent selected from the group comprising or, alternatively, consisting of: (c-i) at least one carrageenan, preferably carrageenan E407, (c-ii) at least one acacia gum, preferably acacia gum E414, (c-iii) at least one fucoidan, and mixtures thereof;

and, optionally, (d) at least one sucrester, preferably sucrester E473; and/or

(e) at least one gelatinised or pre-gelatinised starch of plant origin, preferably pre-gelatinised rice starch.

The following embodiments (FRs) of the solid form formulation of selenium (in short, Se) of the invention (cyclosome or cyclosomal magnesium) are comprised in the present invention:
FRa-Se: Se, (b) and (c) such as (c-i) or (c-ii) or (c-iii); for example Se, (b) and (c-i) or Se, (b) and (c-ii) or Se, (b) and (c-iii).
FRb-Se: Se, (b), (c) such as (c-i) or (c-ii) or (c-iii) and (d); for example Se, (b), (c-i) and (d) or Se, (b), (c-ii) and (d) or Se, (b), (c-iii) and (d).
FRc-Se: Se, (b), (c) such as (c-i) or (c-ii) or (c-iii) and (e); for example Se, (b), (c-i) and (e) or Se, (b), (c-ii) and (e) or Se, (b), (c-iii) and (e).
FRd-Se: Se, (b), (c) such as (c-i) or (c-ii) or (c-iii), (d) and (e); for example Se, (b), (c-i), (d) and (e) or Se, (b), (c-ii), (d) and (e) or Se, (b), (c-iii), (d) and (e).

wherein the components referred to as (a), (b), (c-i), (c-ii), (c-iii), (d) and (e) are as defined in the present invention.

In an embodiment of the invention, said solid form formulation of selenium (referred to as cyclosome or cyclosomal selenium) comprises or, alternatively, consists of:

(a-vi) selenium, preferably selenium (IV), more preferably selenium in the form of sodium selenite;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c) at least one first agent selected from the group comprising or, alternatively, consisting of: (c-i) at least one carrageenan, preferably carrageenan E407, (c-ii) at least one acacia gum, preferably acacia gum E414, (c-iii) at least one fucoidan, and mixtures thereof;

and, optionally, (d) at least one sucrester, preferably sucrester E473; and/or

(e) at least one gelatinised or pre-gelatinised starch of plant origin, preferably pre-gelatinised rice starch.

The following embodiments (FRs) of the solid form formulation of chromium (in short, Cr) of the invention (cyclosome or cyclosomal magnesium) are comprised in the present invention:
FRa-Cr: Cr, (b) and (c) such as (c-i) or (c-ii) or (c-iii); for example Cr, (b) and (c-i) or Cr, (b) and (c-ii) or Cr, (b) and (c-iii).
FRb-Cr: Cr, (b), (c) such as (c-i) or (c-ii) or (c-iii) and (d); for example Cr, (b), (c-i) and (d) or Cr, (b), (c-ii) and (d) or Cr, (b), (c-iii) and (d).
FRc-Cr: Cr, (b), (c) such as (c-i) or (c-ii) or (c-iii) and (e); for example Cr, (b), (c-i) and (e) or Cr, (b), (c-ii) and (e) or Cr, (b), (c-iii) and (e).
FRd-Cr: Cr, (b), (c) such as (c-i) or (c-ii) or (c-iii), (d) and (e); for example Cr, (b), (c-i), (d) and (e) or Cr, (b), (c-ii), (d) and (e) or Cr, (b), (c-iii), (d) and (e).

wherein the components referred to as (a), (b), (c-i), (c-ii), (c-iii), (d) and (e) are as defined in the present invention.

In an embodiment of the invention, said solid form formulation of chromium (referred to as cyclosome or cyclosomal chromium) comprises or, alternatively, consists of:

(a-vii) chromium, preferably chromium (III), more preferably chromium in the form of chromium (III) picolinate or chromium (III) nicotinate or polynicotinate or chromium (III) chloride, even more preferably chromium (III) picolinate;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c) at least one first agent selected from the group comprising or, alternatively, consisting of: (c-i) at least one carrageenan, preferably carrageenan E407, (c-ii) at least one acacia gum, preferably acacia gum E414, (c-iii) at least one fucoidan, and mixtures thereof;

and, optionally, (d) at least one sucrester, preferably sucrester E473; and/or

(e) at least one gelatinised or pre-gelatinised starch of plant origin, preferably pre-gelatinised rice starch.

The following embodiments (FRs) of the solid form formulation of copper (in short, Cu) of the invention (cyclosome or cyclosomal copper) are comprised in the present invention:
FRa-Cu: Cu, (b) and (c) such as (c-i) or (c-ii) or (c-iii); for example Cu, (b) and (c-i) or Cu, (b) and (c-ii) or Cu, (b) and (c-iii).
FRb-Cu: Cu, (b), (c) such as (c-i) or (c-ii) or (c-iii) and (d); for example Cu, (b), (c-i) and (d) or Cu, (b), (c-ii) and (d) or Cu, (b), (c-iii) and (d).
FRc-Cu: Cu, (b), (c) such as (c-i) or (c-ii) or (c-iii) and (e); for example Cu, (b), (c-i) and (e) or Cu, (b), (c-ii) and (e) or Cu, (b), (c-iii) and (e).
FRd-Cu: Cu, (b), (c) such as (c-i) or (c-ii) or (c-iii), (d) and (e); for example Cu, (b), (c-i), (d) and (e) or Cu, (b), (c-ii), (d) and (e) or Cu, (b), (c-iii), (d) and (e).

wherein the components referred to as (a), (b), (c-i), (c-ii), (c-iii), (d) and (e) are as defined in the present invention.

In an embodiment of the invention, said solid form formulation of copper (referred to as cyclosome or cyclosomal copper) comprises or, alternatively, consists of:

(a-i) copper, preferably copper (II), more preferably copper gluconate (Cu(2+)) or copper sulphate (Cu(2+); CuSO₄, for example, anhydrous, pentahydrate or heptahydrate), even more preferably copper gluconate;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c) at least one first agent selected from the group comprising or, alternatively, consisting of: (c-i) at least one carrageenan, preferably carrageenan E407, (c-ii) at least one acacia gum, preferably acacia gum E414, (c-iii) at least one fucoidan, and mixtures thereof;

and, optionally, (d) at least one sucrester, preferably sucrester E473; and/or

(e) at least one gelatinised or pre-gelatinised starch of plant origin, preferably pre-gelatinised rice starch.

Copper gluconate: chemical name: copper, bis(D-gluconate-O1,O2), synonym: Copper D-gluconate, example CAS No: 527-09-3, brute formula: C12H22CuO14, formula weight: 453.84.

Forming an object of the present invention is a solid form formulation of at least one vitamin (in short, formulation of at least one vitamin of the invention or formulation of the invention or, alternatively, cyclosome or cyclosomal vitamin) comprising or, alternatively, consisting of:

(a) at least one vitamin, wherein said vitamin is selected from the group comprising or, alternatively, consisting of: (a-i) at least one vitamin of group B (for example, B12, B9, B6, B3 or B2), preferably vitamin B12, (a-II) a vitamin C, (a-Ill) a vitamin D, preferably D3, (a-IV) a vitamin E, and (a-V) a vitamin A;

(b) at least one phospholipid, preferably a phosphoglyceride, more preferably a phosphatidylcholine or lecithin;

(c) at least one first agent (a polysaccharide) selected from (c-i) at least one carrageenan and/or (c-ii) at least one acacia gum;

and, optionally, (d) at least one fatty acid carbohydrate ester (alternatively referred to as sucrester);

and, optionally, (e) at least one starch of plant origin, preferably gelatinised or pre-gelatinised, for example rice starch.

An example of said solid form formulation of at least one vitamin comprises or, alternatively, consists of:

(a) at least one vitamin selected from: (a-I) vitamin B12, (a-II) vitamin C, (a-Ill) vitamin D, preferably D3, (a-IV) vitamin E;

(b) a lecithin, preferably a sunflower, corn or soy lecithin (preferably sunflower (E322));

(c-i) a carrageenan (e.g. E407) or (c-ii) an acacia gum (e.g. E414);

and, optionally, (d) a sucrester (e.g. sucrester E473);

and, optionally, (e) a gelatinised or pre-gelatinised starch of plant origin, preferably pre-gelatinised rice starch.

An example of said solid form formulation of at least one vitamin comprises or, alternatively, consists of:

(a) at least one vitamin selected from: (a-I) vitamin B12, (a-II) vitamin C, (a-Ill) vitamin D, preferably D3, (a-IV) vitamin E;

(b) a lecithin, preferably a sunflower, corn or soy lecithin (preferably sunflower (E322));

(c-i) a carrageenan (e.g. E407) or (c-ii) an acacia gum (e.g. E414);

(d) a sucrester (e.g. sucrester E473);

and, optionally, (e) a gelatinised or pre-gelatinised starch of plant origin, preferably pre-gelatinised rice starch.

An example of said solid form formulation of at least one vitamin comprises or, alternatively, consists of:

(a) at least one vitamin selected from: (a-I) vitamin B12, (a-II) vitamin C, (a-Ill) vitamin D, preferably D3, (a-IV) vitamin E;

(b) a lecithin, preferably a sunflower, corn or soy lecithin (preferably sunflower (E322));

(c-i) a carrageenan (e.g. E407) or (c-ii) an acacia gum (e.g. E414);

(d) a sucrester (e.g. sucrester E473); and

(e) a gelatinised or pre-gelatinised starch of plant origin, preferably pre-gelatinised rice starch.

The following embodiments (FRs) of the solid form formulation of vitamin B12 (in short, vit B12) of the invention (cyclosome or cyclosomal vitamin B12) are comprised in the present invention:
FRa-vit B12: vit B12, (b) and (c), such as (c-i) or (c-ii); for example vit B12, (b) and (c-i) or vit B12, (b) and (c-ii);
FRb-vit B12: vit B12, (b), (c) (such as (c-i) or (c-ii)) and (d); for example vit B12, (b), (c-i) and (d) or vit B12, (b), (c-ii) and (d);
FRc-vit B12: vit B12, (b), (c) (such as (c-i) or (c-ii)) and (e); for example vit B12, (b), (c-i) and (e) or vit B12, (b), (c-ii) and (e);
FRd-vit B12: vit B12, (b), (c) (such as (c-i) or (c-ii)), (d) and (e); for example vit B12, (b), (c-i), (d) and (e) or vit B12, (b), (c-ii), (d) and (e);

wherein the components referred to as (a), (b), (c-i), (c-ii), (d) and (e) are as defined in the present invention.

In an embodiment of the invention, said solid form formulation of vitamin B12 (referred to as cyclosome or cyclosomal vitamin B12) comprises or, alternatively, consists of:

(a-i) vitamin B12 (cobalamin) (example of CAS No. 68-19-9);

(b) at least one phospholipid, preferably a phosphatidylcholine or lecithin, for example a lecithin (E322) selected from sunflower lecithin, corn lecithin, soy lecithin and mixtures thereof (preferably, sunflower lecithin);

(c) a polysaccharide selected from (c-i) at least one carrageenan (for example E407) or (c-ii) at least one acacia gum (for example E414) and mixtures thereof;

and, optionally, (d) at least one sucrester (for example E473);

and, optionally, (e) at least one gelatinised or pre-gelatinised starch of plant origin preferably pre-gelatinised rice starch.

The following embodiments (FRs) of the solid form formulation of vitamin C (in short, vit C) of the invention (cyclosome or cyclosomal vitamin C) are comprised in the present invention:
FRa-vit C: vit C, (b) and (c), such as (c-i) or (c-ii); for example vit C, (b) and (c-i) or vit C, (b) and (c-ii);
FRb-vit C: vit C, (b), (c) (such as (c-i) or (c-ii)) and (d); for example vit C, (b), (c-i) and (d) or vit C, (b), (c-ii) and (d);
FRc-vit C: vit C, (b), (c) (such as (c-i) or (c-ii)) and (e); for example vit C, (b), (c-i) and (e) or vit C, (b), (c-ii) and (e);
FRd-vit C: vit C, (b), (c) (such as (c-i) or (c-ii)), (d) and (e); for example vit C, (b), (c-i), (d) and (e) or vit C, (b), (c-ii), (d) and (e);

wherein the components referred to as (a), (b), (c-i), (c-ii), (d) and (e) are as defined in the present invention.

In an embodiment of the invention, said solid form formulation of vitamin C (referred to as cyclosome or cyclosomal vitamin C) comprises or, alternatively, consists of:

(a-i) vitamin C (cobalamin) (example of CAS No. 68-19-9);

(b) at least one phospholipid, preferably a phosphatidylcholine or lecithin, for example a lecithin (E322) selected from sunflower lecithin, corn lecithin, soy lecithin and mixtures thereof (preferably, sunflower lecithin);

(c) a polysaccharide selected from (c-i) at least one carrageenan (for example E407) or (c-ii) at least one acacia gum (for example E414) and mixtures thereof;

and, optionally, (d) at least one sucrester (for example E473);

and, optionally, (e) at least one gelatinised or pre-gelatinised starch of plant origin preferably pre-gelatinised rice starch.

The following embodiments (FRs) of the solid form formulation of vitamin D (in short, vit D) of the invention (cyclosome or cyclosomal vitamin D) are comprised in the present invention:
FRa-vit D: vit D, (b) and (c), such as (c-i) or (c-ii); for example vit D, (b) and (c-i) or vit D, (b) and (c-ii);
FRb-vit D: vit D, (b), (c) (such as (c-i) or (c-ii)) and (d); for example vit D, (b), (c-i) and (d) or vit D, (b), (c-ii) and (d);
FRc-vit D: vit D, (b), (c) (such as (c-i) or (c-ii)) and (e); for example vit D, (b), (c-i) and (e) or vit D, (b), (c-ii) and (e);
FRd-vit D: vit D, (b), (c) (such as (c-i) or (c-ii)), (d) and (e); for example vit D, (b), (c-i), (d) and (e) or vit D, (b), (c-ii), (d) and (e);

wherein the components referred to as (a), (b), (c-i), (c-ii), (d) and (e) are as defined in the present invention.

In an embodiment of the invention, said solid form formulation of vitamin D (referred to as cyclosome or cyclosomal vitamin D) comprises or, alternatively, consists of:

(a-i) vitamin D (cobalamin) (example of CAS No. 68-19-9);

(b) at least one phospholipid, preferably a phosphatidylcholine or lecithin, for example a lecithin (E322) selected from sunflower lecithin, corn lecithin, soy lecithin and mixtures thereof (preferably, sunflower lecithin);

(c) a polysaccharide selected from (c-i) at least one carrageenan (for example E407) or (c-ii) at least one acacia gum (for example E414) and mixtures thereof;

and, optionally, (d) at least one sucrester (for example E473);

and, optionally, (e) at least one gelatinised or pre-gelatinised starch of plant origin preferably pre-gelatinised rice starch.

The following embodiments (FRs) of the solid form formulation of vitamin E (in short, vit E) of the invention (cyclosome or cyclosomal vitamin E) are comprised in the present invention:
FRa-vit E: vit E, (b) and (c), such as (c-i) or (c-ii); for example vit E, (b) and (c-i) or vit E, (b) and (c-ii);
FRb-vit E: vit E, (b), (c) (such as (c-i) or (c-ii)) and (d); for example vit E, (b), (c-i) and (d) or vit E, (b), (c-ii) and (d);
FRc-vit E: vit E, (b), (c) (such as (c-i) or (c-ii)) and (e); for example vit E, (b), (c-i) and (e) or vit E, (b), (c-ii) and (e);
FRd-vit E: vit E, (b), (c) (such as (c-i) or (c-ii)), (d) and (e); for example vit E, (b), (c-i), (d) and (e) or vit E, (b), (c-ii), (d) and (e);

wherein the components referred to as (a), (b), (c-i), (c-ii), (d) and (e) are as defined in the present invention.

In an embodiment of the invention, said solid form formulation of vitamin E or (referred to as cyclosome or cyclosomal vitamin E) comprises or, alternatively, consists of:

(a-i) vitamin E (cobalamin) (example of CAS No. 68-19-9);

(b) at least one phospholipid, preferably a phosphatidylcholine or lecithin, for example a lecithin (E322) selected from sunflower lecithin, corn lecithin, soy lecithin and mixtures thereof (preferably, sunflower lecithin);

(c) a polysaccharide selected from (c-i) at least one carrageenan (for example E407) or (c-ii) at least one acacia gum (for example E414) and mixtures thereof;

and, optionally, (d) at least one sucrester (for example E473);

and, optionally, (e) at least one gelatinised or pre-gelatinised starch of plant origin preferably pre-gelatinised rice starch.

Phospholipids are phosphate-containing lipids. The molecules of this class of organic compounds have a water-soluble polar head (i.e., water-soluble and insoluble in apolar solvents) based on phosphate and a hydrophobic non-water-soluble apolar tail (i.e., water-insoluble and soluble in apolar solvents), for this reason they are referred to as amphipathic molecules.

Phosphoglycerides (also called glycerophospholipids) represent the most important class of phospholipids.

Glycerophospholipids (or phosphoglycerides) are all derived from sn-glycerol-3-phosphate, in which glycerol (CH₂OH—CHOH—CH₂OH) is esterified in position 3 with orthophosphoric acid (H₃PO₄). In glycerophospholipids, glycerol is esterified in position 2 with a fatty acid, while in position 1 different classes of compounds can be bound; since carbon 2 is asymmetric, there are two possible stereoisomers: L and D. In nature glycerophospholipids all belong to the L series.

Depending on the nature of the molecule that binds to the position 1 of glycerol, three subclasses of phosphoglycerides can be distinguished: 1,2-di-acyl-phospholipids, 1-alkyl-2-acyl-phospholipids, 1-alkenyl-2-acyl-phospholipids.

Diacyl-phospholipids (phosphoglycerides) derive from the structure of triglycerides, where a fatty acid is replaced by a phosphate group which gives the molecule a negative charge and therefore polarity; this molecule has the generic name phosphatide. A more complex organic molecule, generally serine, choline, ethanolamine, inositol or a single hydrogen atom, is bound to the phosphate group through an ester bond, giving rise to a phospholipid called phosphatidylserine, phosphatidylcholine (or lecithin), phosphatidylethanolamine, phosphatidylinositol or phosphatidic acid, respectively.

Diacyl-phospholipids are characterised by a water-soluble polar head, which dissolves well in water, while the two saturated fatty acids represent the two apolar tails, which are not water-soluble but lipophilic.

The term lecithin is used to indicate a class of chemical compounds present in animal and plant tissues (particularly in the egg yolk). Chemically, a lecithin is a phosphatidylcholine ((R)-1-oleoyl-2-palmitoyl-phosphatidylcholine) or, alternatively, a lecithin comprises phosphatidylcholine as the primary component. A phosphatidylcholine is a phosphoglyceride in which phosphatidic acid is esterified with choline. Phosphatidic acids represent the simplest phosphoglycerides, formally produced by the esterification of glycerol in position 1 and 2 with fatty acids and in position 3 with orthophosphoric acid.

Lecithin is a natural emulsifier due to its chemical/physical properties, and it has a secondary antioxidant function, given that it is rich in natural antioxidant substances.

A lecithin E322 is a food additive (emulsifier). The term “E322” indicates that lecithin is a food additive permitted by European legislation and regulated by the Italian Ministerial Decree N° D.M. 1996.

Directive 2008/84/EC of 27 August 2008 (published in the Official Journal of the European Union No L253) lays down the purity criteria which a lecithin must meet in order to be considered to meet food quality standards (lecithin E322): insoluble in acetone (basically the active part of lecithin):: 60% min.; moisture: 2% max.; acidity number: 35 max.; peroxides number: 10 max.; insoluble in toluene (the impurities basically): 0.3% max.

Advantageously, the (b) phospholipids of the present invention, comprised in the formulation of at least one mineral and/or in the formulation of at least one vitamin of the present invention together with (a), (c) such as (c-i) or (c-ii) or (c-iii) and, optionally, (d) and/or (e), are phosphoglycerides selected from 1,2-diacyl-phospholipids, 1-alkyl-2-acylphospholipids, 1-alkenyl-2-acylphospholipids, preferably diacyl-phospholipids.

In an embodiment of the present invention, said at least one phospholipid (b), comprised in the formulation of at least one mineral and/or in the formulation of at least one vitamin of the invention together with (a), (c) such as (c-i) or (c-ii) or (c-iii) and, optionally, (d) and/or (e), is a phosphoglyceride, preferably a diacyl-phospholipid, more preferably selected from the group of diacyl-phospholipids comprising or, alternatively, consisting of: phosphatidylcholine or lecithin, phosphatidylserine, phosphatidylethanolamine, phosphatidylinositol, phosphatidic acid and mixtures thereof; preferably phosphatidylcholine or lecithin (E322); more preferably sunflower lecithin (E322), corn lecithin (E322) or soy lecithin (E322); even more preferably, allergen free lecithin (E322), for example allergen free sunflower lecithin (E322).

Advantageously, said lecithin (E322) is a powdered or granular lecithin (E322), preferably a powdered or granular sunflower lecithin (E322), corn lecithin (E322) or soy lecithin (E322), even more preferably a powdered or granular allergen free sunflower lecithin (E322), corn lecithin (E322) or soy lecithin (E322). The term “allergen free” means that it does not have any allergen residues.

When said (b) phosphatidylcholine or lecithin (E322) is a powdered or granular lecithin, the lecithin may, for example, have a % by weight water content comprised in a range from 1.5% to 4.5% with respect to the weight of lecithin, preferably from 2% to 4%, even more preferably from 2.5% to 3.5%.

When said (b) phosphatidylcholine or lecithin is a sunflower lecithin E322, preferably powdered or granular, lecithin may have, for example, a % by weight glucose content comprised in the range from 20% to 60% with respect to the weight of lecithin, preferably from 30% to 50%, for example about 45%. A (b) sunflower lecithin E322, preferably in form of powder or granules, that can be used in the context of the present invention may have the following composition at a % by weight (chemical/physical analysis): sunflower lecithin from 40% to 50%, carbohydrates from 40% to 50% (for example about 42%), proteins from 6% to 10%, ashes from 3% to 8%, moisture from 2% to 5% and another flowing agent from 0.5% to 1.5%.

In an embodiment of the present invention, said (b) lecithin, comprised in the formulation of at least one mineral and/or in the formulation of at least one vitamin of the invention together with (a), (c) such as (c-i) or (c-ii) or (c-iii) and, optionally, (d) and/or (e), does not comprise or, alternatively, it does not consist of a decomposed or hydrolysed lecithin, and it does not comprise or, alternatively, it does not consist of an enzymatically decomposed or hydrolysed lecithin.

In other words, said (b) lecithin, comprised in the formulation of at least one mineral and/or at least one vitamin of the invention together with (a), (c) such as (c-i) or (c-ii) or (c-iii) and, optionally, (d) and/or (e), is an undecomposed or enzymatically hydrolysed lecithin (E322).

Preferably, in the formulation according to the present invention (comprising (a) a mineral and/or a vitamin, (b), (c) such as (c-i) or (c-ii) and, optionally, (d) and/or (e)), the (c)+(d)]:(b) weight ratio (i.e., the total weight of a polysaccharide (i.e. carrageenan or acacia gum) and sucrester:a phospholipid (preferably a lecithin, more preferably sunflower lecithin)) is comprised from 50:1 to 10:1 (for example 45:1, 40:1, 35:1, 30:1, 25:1, 20:1, or 15:1), preferably from 40:1 to 10:1, more preferably from 30:1 to 15:1 (for example, about 17:1 or 17:0.6-0.51).

Said [(c)+(d)]:(b) weight ratio may not be obtained for formulations according to the invention comprising calcium.

Preferably, in the solid form formulation of a mineral or in the solid form formulation of a vitamin according to the present invention, components (a), (b), (c) such as (c-i) or (c-ii) and, optionally, (d) and/or (e) are comprised in said formulations in the following amounts expressed as percentage by weight with respect to 100 of total weight of the formulation:
said (b) phospholipid, preferably lecithin or sunflower lecithin (E322), is comprised from 0.05% to 15%, preferably from 0.1% to 5%, more preferably from 0.1% to 2%, for example about 0.5-1.0%;
the sum of (c) first agent (a polysaccharide, such as (c-i) carrageenan or (c-ii) acacia gum) and (d) sucrester (sucrester optionally present in the formulation) is comprised from 1% to 50% (for example, 10%, 20%, 30% or 40%), preferably from 5% to 35%, more preferably from 10% to 25%, for example 15%-20% (such as 16%, 17%, or 18%); and
said (a) at least one mineral (in the form of salt, oxide or complex) and/or at least one vitamin vary as the weight thereof varies;
said (e) optional starch varies with the variation of said percentage of mineral and/or vitamin.

According to a preferred example, the solid form formulation of a mineral and/or the solid form formulation of a vitamin of the invention, comprising (a), (b), (c) such as (c-i) or (c-ii) and, optionally, (d) and/or (e), comprises the following amounts expressed as percentage by weight with respect to 100 of total weight of the formulation:
said (b) phospholipid, preferably lecithin or sunflower lecithin (E322), from about 0.5-1.0%;
the sum of (c) first agent (a polysaccharide, such as (c-i) carrageenan or (c-ii) acacia gum) and (d) sucrester (optionally present sucrester) from 15%-20% (such as 16%, 17%, or 18%); and
said (a) at least one mineral (in the form of salt, oxide or complex) and/or at least one vitamin vary as the weight thereof varies;
said (e) optional starch varies with the variation of said percentage of mineral and/or vitamin.

In 100 parts by weight of said sum of (c) first agent (a polysaccharide, such as (c-i) carrageenan (c-ii) acacia gum) and (d) sucrester (optionally present sucrester), said (c) first agent is comprised in percentage by weight from 1% to 100% (for example, 5%, 10%, 15%, 20%, 25%, 30%, 35%, 40%, 45%, 50%, 55%, 60%, 65%, 70%, 75%, 80%, 85%, 90%, or 95%) and said (d) sucrester varies from 0% (absent) to 99% (for example, 5%, 10%, 15%, 20%, 25%, 30%, 35%, 40%, 45%, 50%, 55%, 60%, 65%, 70%, 75%, 80%, 85%, 90%, or 95%); preferably said (c) first agent from 50% to 95% and said (d) sucrester from 5% to 50% (for example (c) about 50% and (d) about 50%), or said (c) first agent from 70% to 95% and said (d) sucrester from 5% to 30% (for example, (c) 70%-80% and (d) 20%-30%, or (c) about 75% and (d) about 25%), or said (c) first agent from 5% to 30% and said (d) sucrester from 70% to 95% (for example, (c) 20%-30% and (d) 70%-80%, or (c) about 25% and (d) about 75%).

Advantageously, in the formulations of at least one mineral and/or in the formulations of at least one vitamin according to the present invention, in 100 parts by weight of said sum of (c) first agent (a polysaccharide, such as (c-i) carrageenan or (c-ii) acacia gum) and (d) sucrester, said first agent (c) is about 75% and said sucrester (d) is about 25%.

In an embodiment of the solid form formulation of said at least one mineral of the invention, components (a), (b), (c) such as (c-i) or (c-ii) or (c-iii) and, optionally, (d) and/or (e) are present in said formulation in the following amounts expressed as % by weight with respect to the total weight of the formulation of said at least one mineral:
said (a) mineral element (cation of the mineral) varies according to the mineral as follows:
   Mg(II) or magnesium metal is comprised in a range from 5% to 80%, preferably from 10% to 60%, more preferably from 30% to 40%; for example, magnesium oxide about 53-54% corresponding to about 32-38% of Mg(II) element;
   Ca(II) or calcium metal is comprised in a range from 5% to 80%, preferably from 10% to 60%, more preferably from 30% to 40%; for example, tricalcium phosphate about 97% corresponding to about 31-37% of Ca(II) element;
   Fe(III) or iron metal is comprised in a range from 1% to 40%, preferably from 1% to 30%, more preferably from 5% to 20%; for example iron (III) pyrophosphate about 44-45% which corresponds to about 10-12% of Fe(III) element;
   Zn(II) or zinc metal is comprised in a range from 10% to 80%, preferably from 20% to 70%, more preferably from 30% to 60%; for example, zinc oxide about 53-54% corresponding to about 40-50% of Zn(II) element;
   I(V) or iodine metal is comprised in a range from 0.01% to 15%, preferably from 0.05% to 10%, more preferably from 0.1% to 3%; for example, sodium iodate about 1-2% corresponding to about 0.9-1.1% of I(V) element;
   Se(IV) or selenium metal is comprised in a range from 0.01% to 15%, preferably from 0.05% to 10%, more preferably from 0.1% to 3%; for example, sodium selenite about 2.0-2.5% corresponding to about 0.9-1.2% of Se(IV) element;
   Cr(III) or chromium metal is comprised in a range from 1% to 40%, preferably from 3% to 20%, more preferably from 5% to 15%; for example about 9+1% di Cr(III) given by chromium (III) picolinate;
   Cu(II) or metal copper is comprised in a range from 1% to 30%, preferably from 1% to 20%, more preferably from 1% to 10%; for example about 6+1% of Cu(II) given by copper (II) gluconate;
said (b) phospholipid, preferably phosphoglyceride, more preferably phosphatidylcholine or lecithin, even more preferably solid lecithin (E322) or solid sunflower lecithin (E322), is comprised in a range from 0.05% to 15%, preferably from 0.1% to 5%, more preferably from 0.1% to 2%, for example about 0.6±0.5% or 1.0±0.5%;
said (c) first agent, in the form of (c-i) carrageenan or (c-ii) acacia gum or (c-iii) fucoidan or a mixture thereof, is comprised in a range from 1% to 50% (for example, 3%, 5%, 7%, 10%, 12%, 15%, 17%, 20%, 25%, 30%, or 40%), preferably from 1% to 35%, more preferably from 1% to 20% or from 1% to 10% or from 10% to 20%, for example about 4-5+1%, 12-13+1% or 17-18+1%;
if present, said (d) sucrester (E473) is comprised in a range from 1% to 50% (for example, 3%, 5%, 7%, 10%, 12%, 15%, 17%, 20%, 25%, 30%, or 40%), preferably from 1% to 35%, more preferably from 1% to 20% or from 1% to 10% or from 10% to 20%, for example about 4-5+1%, 12-13+1% or 17-18+1%; or, alternatively, absent (0%);
if present, said starch (e), preferably pre-gelatinised rice starch, is comprised in a range from 10% to 85%, preferably from 15% to 60%, more preferably from 25% to 50%, for example about 37-38+1%.

In an embodiment of the solid form formulation of a vitamin of the invention (vitamin C, B12 or E), components (a), (b), (c) (such as (c-i) or (c-ii)) and, optionally, (d) and/or (e) are present in said formulation in the following amounts expressed as percentage by weight with respect to the total weight of the formulation:
said (a) vitamin C or vitamin B12 or vitamin E is comprised in a range from 20% to 80% (for example, 25%, 35%, 40%, 45% 50%, 55%, 60%, 65%, 70%, or 75%) preferably from 45% to 60% (for example, about 50-55%);
said (b) phospholipid, preferably phosphoglyceride, more preferably phosphatidylcholine or lecithin, even more preferably solid lecithin (E322) or solid sunflower lecithin (E322), is comprised in a range from 0.05% to 10%, (for example, 0.1%, 0,2%, 0.3%, 0.4%, 0.5%, 0.6%, 0.7%, 0.8%, 0.9%, 1%, 2%, 3%, 4%, 5% or 8%), preferably from about 0.1% to 2% (for example about 1.0±0.1%);
said (c) first agent (a polysaccharide), such as (c-i) carrageenan or (c-ii) acacia gum, or, alternatively, the sum of said first agent (c) and said (d) sucrester (E473), is comprised in a range from 1% to 50% (for example, 3%, 5%, 7%, 10%, 12%, 14%, 16%, 18%, 20%, 25%, 30%, or 40%), preferably from about 10% to 25% (for example from about 15±1 to 20±1%); and
if present, said (e) starch, preferably pre-gelatinised rice starch, is comprised in a range from 5% to 60%, (for example, 10%, 15%, 20%, 25%, 30%, 35%, 40%, 45%, 50%, or 55%), preferably from about 20% to 40% (for example about 30±0.1%).

In an embodiment of the solid form formulation of vitamin D3 of the invention, components (a), (b), (c) (such as (c-i) or (c-ii)) and, optionally, (d) and/or (e) are present in said formulation in the following amounts expressed as a percentage by weight with respect to the total weight of the formulation:
said (a) vitamin D3 (commercial) is comprised in a range from 50% to 95% (for example, 60%, 70%, 75%, 80%, 85%, or 90%), preferably from about 80% to 90% (for example 85%), wherein the amount of pure vitamin D3 (or vitamin D3 as such) is from about 0.10% to 0.25% (for example, 0.12%, 0.14%, 0.16%, 0.18%, 0.20%, 0.22%, or 0.24%), preferably about 0.20±1%;
said (b) phospholipid, preferably phosphoglyceride, more preferably phosphatidylcholine or lecithin, even more preferably solid lecithin (E322) or solid sunflower lecithin (E322), is comprised in a range from 0.05% to 10%, (for example, 0.1%, 0.2%, 0.3%, 0.4%, 0.5%, 0.6%, 0.7%, 0.8%, 0.9%, 1%, 2%, 3%, 4%, 5% or 8%), preferably from about 0.1% to 2% (for example about 0.5±0.1 to 1.0±0.1%);
said (c) first agent (a polysaccharide), such as (c-i) carrageenan or (c-ii) acacia gum, or, alternatively, the sum of said first agent (c) and said (d) sucrester (E473), is comprised in a range from 1% to 50% (for example, 3%, 5%, 7%, 10%, 12%, 14%, 16%, 18%, 20%, 25%, 30%, or 40%), preferably from about 5% to 20% (for example from about 10±1 to 15±1%); and
if present, said (e) starch, preferably pre-gelatinised rice starch, is comprised in a range from 0.05% to 10%, (for example, 0.1%, 0.2%, 0.3%, 0.4%, 0.5%, 0.6%, 0.7%, 0.8%, 0.9%, 1%, 2%, 3%, 4%, 5% or 8%), preferably from about 0.1% to 2% (for example about 1.0±0.1%).

It is clear that, in the solid form formulation of vitamin D3 according to the present invention, the amount of commercial vitamin D3 may vary depending on the amount of pure vitamin D3 (or vitamin D3 as such) comprised in said commercial vitamin D3, so as to have—in the formulation of the invention—a percentage by weight of pure vitamin D3 of about 0.10%-0.25%, preferably about 0.20±1%, with respect to the weight of the formulation.

In an embodiment A of the present invention wherein said (c) is (c-i) carrageenan, said solid form formulation of at least one mineral comprises or, alternatively, consists of:

(a) at least one mineral in the form of a salt or complex of said at least one mineral, wherein said mineral is selected from the group comprising or, alternatively, consisting of: (a-i) magnesium (II), (a-ii) calcium (II), (a-iii) iron (III) or iron (II), (a-iv) zinc (II), (a-v) iodine (V), (a-vi) selenium (IV), (a-vii) chromium (III), (a-viii) copper (II) and mixtures thereof;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c-i) at least one carrageenan, preferably carrageenan E407;

and, optionally, (d) at least one sucrester, preferably sucrester E473;

and/or (e) at least one gelatinised or pre-gelatinised starch of plant origin, preferably pre-gelatinised rice starch.

A preferred example of said embodiment A comprises or, alternatively, consists of:

(a) at least one mineral selected from: (a-i) magnesium (II), (a-ii) calcium (II), (a-iii) iron (III) or iron (II), (a-iv) zinc (II), (a-v) iodine (V), (a-vi) selenium (IV), (a-vii) chromium (III), or (a-viii) copper (II);

(b) a lecithin, preferably a sunflower, corn or soy lecithin (preferably sunflower (E322));

(c-i) a carrageenan, preferably carrageenan E407;

(d) a sucrester (e.g. sucrester E473);

and, optionally, (e) a gelatinised or pre-gelatinised starch of plant origin, preferably pre-gelatinised rice starch.

In a preferred embodiment A of the present invention wherein said (c) is (c-i) carrageenan, said solid form formulation of at least one mineral and/or at least one vitamin comprises or, alternatively, consists of: (a), (b), (c-i), (d) and (e), as defined in embodiment A.

In a further preferred embodiment A of the present invention wherein said (c) is (c-i) carrageenan, said solid form formulation of at least one mineral and/or at least one vitamin comprises or, alternatively, consists of: (a), (b), (c-i), and (e), as defined in embodiment A.

Carrageenan is a food additive (thickener, stabiliser, gelling agent, emulsifier) permitted by European and Italian legislation and classified according to European legislation as a food additive “E407”.

Carrageenan or carrageenin (INCI Name: Carrageenan EU INCI Name: Chrondrus Chrispus Powder) is a product derived from carrageheen, whose name derives from Carragheen in Ireland. Carrageenans are a complex group of linear polysaccharides, often sulphates, extracted from red algae. They are compounds with high molecular weight, i.e. very large molecules, characterised by repeating units of galactose and 3,6-anhydrogalactose (3,6-AG), both sulphates and non-sulphates. The units alternate with alpha 1-3 and beta 1-4 glycosidic bonds. Due to their structure, carrageenans behave like highly flexible molecules which curl to form helical structures. The spatial arrangement of the molecule generally confers them the ability to form a wide variety of different gels at room temperature. There are mainly three commercial classes of carrageenan: lambda, kappa and iota.

Carrageenan essentially consists of calcium, potassium, sodium and magnesium salts, sulfuric esters of polysaccharides which, by hydrolysis, give galactose and 3,6-anhydrogalactose. Carrageenan must not be hydrolysed or otherwise degraded chemically. It is in the form of a powder of coarse to fine consistency, yellowish to colourless in colour and basically odourless.

Carrageenan is a gelatine widely used for food, medicine and industrial purposes (used to clarify honey, beer, for the manufacture of paper, starch and more), especially in Ireland and in Great Britain; it is obtained by boiling two red algae on the rocky coast of the North Atlantic (Chondrus crispus and Gigartina mamitiosa) known by the names Irish moss or carragheen.

An example of (c-i) carrageenan that can be used in the present invention is a carrageenan of the CSW-2 type (Genuvisco®, registered trademark, CP Kelco) standardised with sucrose; CAS 9000-07-1, 57-50-1; according to European standard E407; pH (0.5% solution) 7.0-10.0; loss on drying <=12.0, instantaneous viscosity—high salts <=12; instantaneous viscosity—low salts >=50.

Said (c-i) carrageenan may be obtained according to processes known to the man skilled in the art, for example by extraction from seaweed with water and Ca(OH)₂ at high temperature (e.g. slightly >100° C.), neutralisation and precipitation with ethanol or isopropanol.

In a preferred embodiment of the solid form formulation of said at least one mineral of the invention wherein (c) is (c-i) carrageenan, components (a), (b), (c-I) and, optionally, (d) and/or (e) are present in said formulation in the following amounts expressed as % by weight with respect to the total weight of the formulation of said at least one mineral:
said (a) mineral element (cation of the mineral) varies according to the mineral as follows:
   Mg(II) or magnesium metal is comprised in a range from 5% to 80%, preferably from 10% to 60%, more preferably from 30% to 40%; for example, magnesium oxide about 53-54% corresponding to about 32-38% of Mg(II) element;
   Ca(II) or calcium metal is comprised in a range from 5% to 80%, preferably from 10% to 60%, more preferably from 30% to 40%; for example, tricalcium phosphate about 97% corresponding to about 31-37% of Ca(II) element;
   Fe(III) or iron metal is comprised in a range from 1% to 40%, preferably from 1% to 30%, more preferably from 5% to 20%; for example iron (III) pyrophosphate about 44-45% which corresponds to about 10-12% of Fe(III) element;
   Zn(II) or zinc metal is comprised in a range from 10% to 80%, preferably from 20% to 70%, more preferably from 30% to 60%; for example, zinc oxide about 53-54% corresponding to about 40-50% of Zn(II) element;
   I(V) or iodine metal is comprised in a range from 0.01% to 15%, preferably from 0.05% to 10%, more preferably from 0.1% to 3%; for example, sodium iodate about 1-2% corresponding to about 0.9-1.1% of I(V) element;
   Se(IV) or selenium metal is comprised in a range from 0.01% to 15%, preferably from 0.05% to 10%, more preferably from 0.1% to 3%; for example, sodium selenite about 2.0-2.5% corresponding to about 0.9-1.2% of Se(IV) element;
   Cr(III) or chromium metal is comprised in a range from 1% to 40%, preferably from 3% to 20%, more preferably from 5% to 15%; for example about 9+1% di Cr(III) given by chromium (III) picolinate;
   Cu(II) or metal copper is comprised in a range from 1% to 30%, preferably from 1% to 20%, more preferably from 1% to 10%; for example about 6+1% of Cu(II) given by copper (II) gluconate;
said (b) phospholipid, preferably phosphoglyceride, more preferably phosphatidylcholine or lecithin, even more preferably solid lecithin (E322) or solid sunflower lecithin (E322), is comprised in a range from 0.05% to 15%, preferably from 0.1% to 5%, more preferably from 0.1% to 2%, for example about 0.6+0.5%;
(c-i) carrageenan is comprised in a range from 1% to 50% (e.g. 5%, 10%, 15%, 20%, 25%, 30%, 35%, 40% or 45%), preferably from 1% to 35%, more preferably from 1% to 10% (for example about 4-5+1%) or from 10% to 20% (for example about 12-13+1% o 17-18+1%);
if present, said (d) sucrester (E473) is comprised in a range from 1% to 50% (e.g. 5%, 10%, 15%, 20%, 25%, 30%, 35%, 40% or 45%), preferably from 1% to 35%, more preferably from 10% a 20% (for example about 12-13+1% or 17-18+1%) or from 1% to 10% (for example about 4-5+1%);
if present, said starch (e), preferably pre-gelatinised rice starch, is comprised in a range from 10% to 85%, preferably from 15% to 60%, more preferably from 25% to 50%, for example about 37-38+1%.

In an embodiment B of the present invention wherein said (c) is (c-ii) acacia gum, said solid form formulation of at least one mineral comprises or, alternatively, consists of:

(a) at least one mineral in the form of a salt or complex of said at least one mineral, wherein said mineral is selected from the group comprising or, alternatively, consisting of: (a-i) magnesium (II), (a-ii) calcium (II), (a-iii) iron (III), (a-iv) zinc (II), (a-v) iodine (V), (a-vi) selenium (IV), (a-vii) chromium (III), (a-viii) copper (II) and mixtures thereof;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c-ii) at least one acacia gum, preferably acacia gum E414; more preferably an acacia gum E414 having an average molecular weight comprised in the range from 250,000 to 400,000, more preferably an average molecular weight of about 350,000;

and, optionally, (d) at least one sucrester, preferably sucrester E473;

and/or (e) at least one gelatinised or pre-gelatinised starch of plant origin, preferably pre-gelatinised rice starch.

A preferred example of said embodiment A comprises or, alternatively, consists of:

(a) a mineral selected from: (a-i) magnesium (II), (a-ii) calcium (II), (a-iii) iron (III) or iron (II), (a-iv) zinc (II), (a-v) iodine (V), (a-vi) selenium (IV), (a-vii) chromium (III), or (a-viii) copper (II);

(b) a lecithin, preferably a sunflower, corn or soy lecithin (preferably sunflower (E322));

(c-i) an acacia gum, preferably acacia gum E414;

(d) a sucrester (e.g. sucrester E473);

and, optionally, (e) a gelatinised or pre-gelatinised starch of plant origin, preferably pre-gelatinised rice starch.

In a preferred embodiment B of the present invention wherein said (c) is (c-ii) acacia gum, said solid form formulation of at least one mineral and/or at least one vitamin comprises or, alternatively, consists of: (a), (b), (c-ii), (d) and (e), as defined in embodiment B.

In a further preferred embodiment B of the present invention wherein said (c) is (c-ii) acacia gum, said solid form formulation of at least one mineral and/or at least one vitamin comprises or, alternatively, consists of: (a), (b), (c-ii) and (e), as defined in embodiment B.

acacia gum or gum arabic is a food additive (thickener, stabiliser, emulsifier) permitted by European and Italian legislation and classified according to European legislation as a food additive “E414”.

acacia gum is a natural gum of plant origin referred to as acacia gum, since it is extracted from two species of sub-Saharan acacia: Acacia senegal (L) and Acacia seyal. In particular, acacia gum is a dry exudate obtained from trunks and branches of natural strains of acacias and the like.

Gum arabic is a complex mixture of high molecular weight polysaccharides (Mw 250.000-400.000), the calcium, magnesium and potassium salts thereof and glycoproteins which confer them one of its most important properties: the fact that it is fully edible (edibility). It consists of various components: arabinose, D-galactopyranose, rhamnopyranose, D-glucuronic acid, calcium, magnesium, potassium, sodium, mucilage.

Like almost all gums and resins of plant origin, it is produced by the plant following a natural process of “gummosis” that spontaneously activates to heal an insult (wound) to its surface integrity. It is an excipient used primarily in the food industry as a “stabiliser”, but it also has viscosity control functions in certain inks.

An example of (c-ii) acacia gum that can be used in the present invention is an acacia gum having CAS-No. 232-519-5, EINECS 232-519-5, compliant E414, purity min. 99,9%, loss on drying max 10%, total ash max 4%, viscosity (20° C.) min 60 cps., specific optical rotation 30-60° (commercial example 386° gum arabic manufactured by Chimab, cod. 306045).

Acacia gums are obtained according to methods known to the man skilled in the art comprising the steps of centrifugation, filtering, heating and drying.

In an embodiment C of the present invention wherein said (c) is (c-iii) fucoidan, said solid form formulation of at least one mineral comprises or, alternatively, consists of:

(a) at least one mineral in the form of a salt or complex of said at least one mineral, wherein said mineral is selected from the group comprising or, alternatively, consisting of: (a-i) magnesium (II), (a-ii) calcium (II), (a-iii) iron (III), (a-iv) zinc (II), (a-v) iodine (V), (a-vi) selenium (IV), (a-vii) chromium (III), (a-viii) copper (II) and mixtures thereof;

(b) at least one phospholipid, preferably a phosphoglyceride, more preferably a phosphatidylcholine or lecithin, even more preferably a lecithin (E322) selected from sunflower lecithin, corn lecithin, soy lecithin and mixtures thereof;

(c-iii) at least one fucoidan; preferably a fucoidan having an average molecular weight comprised in the range from 20,000 to about 30,000;

and, optionally, (d) at least one sucrester, preferably sucrester E473;

and/or (e) at least one gelatinised or pre-gelatinised starch of plant origin, preferably pre-gelatinised rice starch.

In a preferred embodiment C of the present invention wherein said (c) is (c-iii) fucoidan, said solid form formulation of at least one mineral and/or at least one vitamin comprises or, alternatively, consists of: (a), (b), (c-iii), (d) and (e), as defined in embodiment C.

In a further preferred embodiment C of the present invention wherein said (c) is (c-iii) fucoidan, said solid form formulation of at least one mineral and/or at least one vitamin comprises or, alternatively, consists of: (a), (b), (c-ii) and (e), as defined in embodiment C.

Fucoidan is a sulfated polysaccharide (average molecular weight about 20,000-30,000) present mainly in various brown algae species such as mozuku, kombu, rovina vescica, wakame and hijiki, from which it is extracted preferably by means methods for low temperature extraction with water and subsequent precipitation with ethanol, according to the methods known to the man skilled in the art.

An example of (c-iii) fucoidan that can be used in the present invention is a fucoidan having CAS-No. 9072-19-9, solubility in H₂OR >95, specific gravity (H₂O=1) 0.45-0.65 g/cm³ (supplied by Zhejiang Sanhe Biotech Co., Ltd.).

A further example of (c-iii) fucoidan that can be used in the present invention is a fucoidan obtained by extraction from Laminaria Japonica, a species of brown algae, by means of low-temperature extraction with water and having the following chemical composition: fucoidan 85% min (UV), organic so₄²⁻ 20% min (GBT16484.12-2006), carbohydrates 60% min, L-Fucose 23% min, alginic acid 31% max; and the following physical characteristics: density (bulk density) 40-60 g/100 ml, total ash 3.0% max (USP<281>), loss on drying 5.0% max (USP<731>).

In a preferred embodiment of the solid form formulation of said at least one mineral of the invention wherein (c) is (c-ii) acacia gum or (c-iii) fucoidan, components (a), (b), (c) such as (c-ii) or (c-iii) and, optionally, (d) and/or (e) are present in said formulation in the following amounts expressed as % by weight with respect to the total weight of the formulation of said at least one mineral:
said (a) mineral element (cation of the mineral) varies according to the mineral as follows:
   Mg(II) or magnesium metal is comprised in a range from 5% to 80%, preferably from 10% to 60%, more preferably from 30% to 40%; for example, magnesium oxide about 53-54% corresponding to about 32-38% of Mg(II) element;
   Ca(II) or calcium metal is comprised in a range from 5% to 80%, preferably from 10% to 60%, more preferably from 30% to 40%; for example, tricalcium phosphate about 97% corresponding to about 31-37% of Ca(II) element;
   Fe(III) or iron metal is comprised in a range from 1% to 40%, preferably from 1% to 30%, more preferably from 5% to 20%; for example iron (III) pyrophosphate about 44-45% which corresponds to about 10-12% of Fe(III) element;
   Zn(II) or zinc metal is comprised in a range from 10% to 80%, preferably from 20% to 70%, more preferably from 30% to 60%; for example, zinc oxide about 53-54% corresponding to about 40-50% of Zn(II) element;
   I(V) or iodine metal is comprised in a range from 0.01% to 15%, preferably from 0.05% to 10%, more preferably from 0.1% to 3%; for example, sodium iodate about 1-2% corresponding to about 0.9-1.1% of I(V) element;
   Se(IV) or selenium metal is comprised in a range from 0.01% to 15%, preferably from 0.05% to 10%, more preferably from 0.1% to 3%; for example, sodium selenite about 2.0-2.5% corresponding to about 0.9-1.2% of Se(IV) element;
   Cr(III) or chromium metal is comprised in a range from 1% to 40%, preferably from 3% to 20%, more preferably from 5% to 15%; for example about 9+1% di Cr(III) given by chromium (III) picolinate;
   Cu(II) or metal copper is comprised in a range from 1% to 30%, preferably from 1% to 20%, more preferably from 1% to 10%; for example about 6+1% of Cu(II) given by copper (II) gluconate;
said (b) phospholipid, preferably phosphoglyceride, more preferably phosphatidylcholine or lecithin, even more preferably solid lecithin (E322) or solid sunflower lecithin (E322), is comprised in a range from 0.05% to 15%, preferably from 0.1% to 5%, more preferably from 0.1% to 2%, for example about 0.6+0.5%;
said first agent (c), in the form of (c-ii) acacia gum or (c-iii) fucoidan (for example fucoidan at 80-85% by weight) or a mixture thereof, is comprised in a range from 1% to 50% (for example, 3%, 5%, 7%, 10%, 12%, 15%, 20%, 17%, 25%, 30%, or 40%) preferably from 1% to 35%, more preferably from 10% to 20%, for example about 12-13+1% or 17-18+1%;
if present, said (d) sucrester (E473) is comprised in a range from 1% to 50% (for example, 3%, 5%, 7%, 10%, 12%, 15%, 17%, 20%, 25%, 30%, or 40%), preferably from 1% to 35%, more preferably from 1% to 10%, for example about 4-5+1% or absent;
if present, said starch (e), preferably pre-gelatinised rice starch, is comprised in a range from 10% to 85%, preferably from 15% to 60%, more preferably from 25% to 50%, for example about 37-38+1%.

In an embodiment of the present invention, said formulation of at least one mineral and/or at least one vitamin comprises or, alternatively, consists of: (a), (b), (c) quale (c-i) o (c-ii) or (c-iii), (d) and, optionally, (e).

In said embodiment of the present invention, said (d) sucrester or fatty acid carbohydrate ester, comprised in the formulation of at least one mineral or in the formulation of at least one vitamin of the invention together with (a), (b), (c) such as (c-i) or (c-ii) or (c-iii) and, optionally, (e), is a sucrester E473, preferably a sucrester E473 comprising at least 50% by weight, preferably from 70% to 90% by weight, with respect to the total weight of the sucrester, of monoesters obtained by esterification of sucrose with one or more fatty acids of plant origin, preferably wherein said fatty acids are selected from stearic acid and/or palmitic acid.

The abbreviation “E473” is used to indicate that sucresters or sucrose fatty acid esters are food additives (emulsifiers) permitted by European legislation and regulated by the Italian Ministerial Decree D.M. 1996.

“Sucresters” are generally obtained from the esterification of the fatty acids or from the trans-esterification of fatty acids methyl esters with carbohydrates (also called saccharides). Sucrose (monosaccharide) and polysaccharides are generally the carbohydrates used. This is why sucresters are also referred to as “sucrose fatty acid esters”. The chemical/physical properties of these compounds depend on the number and the type of esterified fatty acids.

They are essentially emulsifiers and they are added to the compositions so as to determine a greater stabilisation of an aqueous phase with a fatty phase.

For example, a (d) sucrester (E473), that can be used in the context of the present invention, may be a sucrester having an HLB (hydrophilic-lipophilic balance) value comprised in the range from 14 to 18, preferably an HLB value of about 15 or 16.

A (d) sucrester (E473), that can be used in the context of the present invention, may have the following composition by weight: total ester content of at least 90%, of which at least 70% by weight, with respect to the total weight of the sucrester, of monoesters obtained through esterification of sucrose with one or more fatty acids of plant origin, preferably stearic acid and/or palmitic acid; free fatty acid content (such as oleic acid) not exceeding 3%; free sucrose content not exceeding 2%; humidity not exceeding 4%; acid value not exceeding 5. An example of (d) commercial sucrester that can be used in the context of the present invention is: sucrose esters SP70 of the company Chimab S.p.A-Italia.

In an embodiment of the present invention, said (d) sucrester, comprised in the formulation of at least one mineral or in the formulation of at least one vitamin of the invention together with (a), (b), (c) such as (c-i) or (c-ii) or (c-iii), and, optionally, (e), does not comprise or, alternatively, it does not consist of a polyglycerol fatty acid ester.

In an embodiment of the present invention, said formulation of at least one mineral or said formulation of at least one vitamin comprises or, alternatively, consists of: (a), (b), (c) such as (c-i) o (c-ii) o (c-iii), (e) and, optionally, (d).

In said embodiment of the present invention, the formulation of at least one mineral or the formulation of at least one vitamin of the invention further comprises, together with (a), (b), (c) such as (c-i) or (c-ii) or (c-iii), and, optionally, (d), a (e) starch of plant origin, preferably gelatinised or pre-gelatinised; preferably, said starch of plant origin is selected from rice starch and/or corn starch; preferably, said starch of vegetable origin is rice starch (Oryza sativa) or native rice starch, preferably gelatinised or pre-gelatinised; more preferably, said starch of plant origin is pre-gelatinised rice starch.

A pre-gelatinised rice starch (E) that can be used in the context of the present invention may have, for example, the following chemical/physical characteristics: humidity not exceeding 7%; protein content not exceeding 1%; ash content not exceeding 1%; pH (10% solution) comprised from 5.5 to 7.5, density 0.40-0.48 g/cm³; minimum starch content at 97% and fat content not exceeding 0.1%. An example of commercial pre-gelatinised rice starch is AX-FG-P manufactured by Reire Srl-Italia.

In an alternative embodiment of the present invention, the formulation of at least one mineral or the formulation of at least one vitamin of the invention further comprises, together with (a), (b), (c) such as (c-i) or (c-ii) or (c-iii), and, optionally, (d) and/or (e), at least one second agent (f) comprising or, alternatively, consisting of: (f-i) at least one cyclodextrin, preferably an a-cyclodextrin; (f-ii) at least one fatty acid having a number of carbon atoms comprised in the range from C6 to C18, preferably from C12 to C18; (f-iii) at least one chitosan derivative, preferably carboxymethylchitosan; and mixtures thereof.

Forming an object of the present invention is a composition (in short, composition of the invention), preferably in solid form, comprising or, alternatively, consisting of: at least one solid form formulation of a mineral (formulation of at least one mineral of the invention) and/or at least one solid form formulation of a vitamin (formulation of a vitamin of the invention) comprising or, alternatively, consisting of (a), (b), (c) such as (c-i) or (c-ii) or (c-iii) and, optionally, (d) and/or (e) according to any one of the embodiments described in the present invention (for example, FR-Mg da (a) to (d), FR-Ca from (a) to (d), FR-Fe from (a) to (d), FR-Zn from (a) to (d), FR-I from (a) to (d), FR-Se from (a) to (d), FR-Cr from (a) to (d), FR-vit B12 from (a) to (d), FR-vit C from (a) to (d), FR-vit D from (a) to (d), FR-vit E from (a) to (d),) and, optionally, said composition comprises at least one acceptable pharmaceutical or food grade additive and/or excipient.

Said at least one acceptable pharmaceutical or food grade additive and/or excipient may be selected from all the substances known to the man skilled in the art of pharmaceutical or food preparations, such as preservatives, emulsifiers and/or thickeners, such as for example hydroxymethyl cellulose, sweeteners, dyes such as for example dye E171, natural and artificial flavours, antioxidants, stabilisers, fillers, anti-caking agents, such as for example vegetable magnesium stearate, magnesium salts of fatty acids and silicon dioxide, fillers, such as microcrystalline cellulose, and mixtures thereof.

In a first aspect, said composition of the invention may comprise a single solid form formulation of a mineral (such as, Mg, Ca, Fe, Zn, I, Se, Cr or Cu).

In a second aspect, said composition of the invention may comprise a single solid form formulation of a vitamin (for example, vitamin B12, C, D3 or E).

In a third aspect, said composition of the invention may comprise two, three, or four solid form formulations of said minerals (such as, Mg, Ca, Fe, Zn, I, SE, Cr and/or Cu).

In a fourth aspect, said composition of the invention may comprise two, three, or four solid form formulations of said vitamins (for example, vitamin B12, C, D3 and/or E).

In a fifth aspect, said composition of the invention may comprise a single solid form formulation of a mineral (such as, Mg, Ca, Fe, Zn, I, Se, Cr or Cu) and a single solid form formulation of a vitamin (for example, vitamin B12, C, D3 or E).

In a sixth aspect, said composition of the invention may comprise two, three, or four solid form formulations of said minerals (such as, Mg, Ca, Fe, Zn, I, Se, Cr and/or Cu) and a single solid form formulation of a vitamin (for example, vitamin B12, C, D3 or E).

In a seventh aspect, said composition of the invention may comprise a single solid form formulation of a mineral (such as, Mg, Ca, Fe, Zn, I, Se, Cr or Cu) and two, three, or four solid form formulations of said vitamins (for example, vitamin B12, C, D3 and/or E).

In an eighth aspect, said composition of the invention may comprise two, three, or four solid form formulations of said minerals (such as, Mg, Ca, Fe, Zn, I, Se, Cr and/or Cu) and two, three, or four solid form formulations of said vitamins (for example, vitamin B12, C, D3 and/or E).

Examples of compositions according to the present invention comprising at least one cyclosomal mineral and/or at least one cyclosomal vitamin or, alternatively, a plurality of cyclosomal minerals, or alternatively, a plurality of cyclosomal vitamins are as follows:

iron+at least one vitamin selected from vitamin C, D3, B12, E and a mixture thereof; iron+vitamin C; iron+vitamin D3; iron+vitamin C+vitamin D3; iron+vitamin C+vitamin B12; iron+vitamin D3+vitamin B12; iron+vitamin C+vitamin D3+vitamin B12;

iron+chromium+at least one mineral selected from Mg, Ca, Zn, I, Se, Cr, Cu and a mixture thereof; iron+chromium+vitamin D3+vitamin B12; iron+chromium+vitamin D3+vitamin B12+vitamin C; iron+chromium+vitamin C+vitamin B12; iron+chromium+vitamin C+vitamin D3;

calcium+at least one vitamin selected from vitamin C, D3, B12, E and a mixture thereof; calcium+vitamin D3; calcium+vitamin D3+at least one vitamin selected from vitamin C, B12, E and a mixture thereof;

calcium+magnesium+at least one vitamin selected from vitamin C, D3, B12, E and a mixture thereof;

magnesium+calcium+vitamin D3; magnesium+calcium+vitamin C; magnesium+calcium+vitamin C+vitamin D3; magnesium+calcium+vitamin D3+at least one vitamin selected from vitamin C, B12, E and a mixture thereof;

magnesium+at least one vitamin selected from vitamin C, D3, B12, E and a mixture thereof;

zinc+at least one vitamin selected from vitamin C, D3, B12, E and a mixture thereof; zinc+vitamin C+vitamin D; zinc+vitamin C+vitamin D3+vitamin B12 e/o vitamin E;

selenium+at least one vitamin selected from vitamin C, D3, B12, E and a mixture thereof; selenium+vitamin E; selenium+vitamin E+at least one vitamin selected from vitamin C, D3, B12 and a mixture thereof;

selenium+iodine+at least one vitamin selected from vitamin C, D3, B12, E and a mixture thereof;

selenium+iodine+vitamin E; selenium+iodine+vitamin E+at least one vitamin selected from vitamin C, D3, B12 and a mixture thereof;

iodine+at least one vitamin selected from vitamin C, D3, B12, E and a mixture thereof;

chromium+at least one vitamin selected from vitamin C, D3, B12, E and a mixture thereof;

copper+at least one vitamin selected from vitamin C, D3, B12, E and a mixture thereof;

chromium+copper+zinc; chromium+copper; chromium+zinc; copper+zinc; chromium+copper+zinc+at least one vitamin selected from vitamin C, D3, B12, E and a mixture thereof

vitamin C+at least one vitamin selected from vitamin D3, B12, E and a mixture thereof (e.g. D3+B12, D3+E, B12+E, D 3+B12+E).

Preferred examples of compositions according to the present invention comprising at least one cyclosomal mineral and at least one cyclosomal vitamin or, alternatively, a plurality of cyclosomal minerals, are as follows:

iron+vitamin C; iron+vitamin D; iron+vitamin C+vitamin D; iron+vitamin C+vitamin B12; iron+chromium+vitamin D+vitamin B12; calcium+vitamin D; magnesium+calcium+vitamin D; zinc+vitamin C+vitamin D; selenium+vitamin E; selenium+iodine+vitamin E, chromium+copper+zinc.

In the composition according to the present invention wherein a plurality of minerals and/or vitamins are present, each mineral and each vitamin is formulated with (b), (c) and, optionally, (d) and/or (e) according to the present invention preferably independently (La separate and independent cyclosomal formulations for each mineral or vitamin).

Advantageously, the composition of the invention, comprising at least one formulation of a mineral and/or a vitamin of the invention (comprising or, alternatively, consisting of (a), (b), (c) such as (c-i) or (c-ii) or (c-iii) and, optionally, (d) and/or (e) according to any one of the described embodiments), further comprises at least one or more further active components selected from the group comprising or, alternatively, consisting of:

(g) at least one vitamin selected from the group of vitamins comprising or, alternatively, consisting of: (g-i) a vitamin of group C, (g-ii) a vitamin of group E, (g-iii) a vitamin of group B, preferably vitamin B6 and/or B9 and/or B12, (g-iv) a vitamin of group D, preferably vitamin D3, and mixtures thereof; preferably (g-i) vitamin C (L-ascorbic acid and/or sodium L-ascorbate);
(h) at least one organic salt or an inorganic salt, preferably selected from the group comprising or, alternatively, consisting of: (h-i) magnesium glycinate, (h-ii) selenium methionine, (h-iii) zinc gluconate and mixtures thereof;
(l) at least one antioxidant, preferably selected from the group comprising or, alternatively, consisting of: (I-i) N-acetyl cysteine (NAC), (I-ii) Coenzyme Q10 (CoQ10), (g-iii) acetyl-L-carnitine (ALC) and the mixtures thereof; and
(m) folic acid;
(n) amino acids, for example phenylalanine, isoleucine, histidine, leucine, lysine, methionine, threonine, tryptophan, valine, arginine, cysteine and tyrosine.

Preferably, each of said vitamins (for example, vitamin B12, C, D3 and/or E) is present in the composition of the invention in the form of formulation according to the invention, wherein said formulation comprises (a) said at least one vitamin, (b) a phospholipid (preferably lecithin), (c) a first agent (preferably (c-i) carrageenan or (c-ii) acacia gum) and, optionally, (d) a sucrester and/or (e) a starch.

Advantageously, if present, each individual vitamin (g) and/or salt (h) is present in the composition of the invention at an amount equal to 100% RDA (recommended dietary allowance).

Advantageously, if present, each single antioxidant substance (l) is present in the composition of the invention at an amount equal to 100 mg/day.

In a preferred embodiment of the invention, the compositions of the invention are in solid form, for example in solid form as such or in mouth-soluble solid form (or mouth-dispersible solid form, which dissolve in the oral cavity) or in water-dispersible solid form.

Alternatively, the compositions of the invention may be in liquid form, for example in the form of solution, dispersion or suspension of a solid in a liquid, or in semi-solid form, for example in form of creams or gels or soft-gels.

In a preferred embodiment of the invention, the compositions of the invention are formulated for oral administration (in short, per os).

Advantageously, the compositions of the invention are in solid form as such or mouth-soluble or water-dispersible for oral administration, such as for example powder, granules, microgranules, flakes, tablets or capsules.

If the compositions of the invention are in tablet form, said tablet may have a weight comprised in the range from 200 mg to 2000 mg, for example a hard tablet from 800 mg to 1000 mg.

Said tablets may be coated or filmed with one or more coating layers or films capable of going past the gastric barrier. Said coating may comprise bee wax or a sugar-based solution.

If the compositions of the invention are in the form of a capsule, said capsule may be of hard gelatine or soft gelatine or soft-gel; preferably a gelatine capsule may have a weight comprised in the range from 100 mg to 1500 mg, more preferably about 800 mg.

The composition of the invention, comprising said solid form formulation of at least one mineral of the invention according to any one of the embodiments described in the present invention, may be a pharmaceutical composition, a medical device composition, a dietary supplement, a food or novel food or nutraceutical composition.

In the context of the present invention, the expression “medical device” is used in the meaning according to the Italian Legislative Decree n° 46 dated 24 Feb. 1997 or according to the new Medical Device Regulation (EU) 2017/745 (MDR).

Forming an object of the present invention is said solid form formulation of at least one mineral and/or said solid form formulation of at least one vitamin of the invention, comprising or, alternatively, consisting of (a), (b), (c) such as (c-i) or (c-ii) or (c-iii) and, optionally, (d) and/or (e) according to any one of the described embodiments, and the compositions of the invention comprising said formulations of at least one mineral and/or at least one vitamin of the invention, according to any one of the described embodiments, for use as medicament, both as a primary medicament and as a medicament for supporting other medicaments (adjuvant).

Furthermore, forming an object of the present invention are the compositions and formulations of at least one mineral of the present invention (such as magnesium, calcium, iron, zinc, iodine, selenium, copper or chromium) and/or of at least one vitamin of the present invention (such as vitamin A, of group B (e.g. B12), C, D (e.g. D3) and/or E), according to any one of the described embodiments, for use in a method for the preventive and/or curative and/or adjuvant treatment of an insufficiency or deficiency of said at least one mineral and/or of said at least one vitamin, and of diseases, symptoms and/or disorders related to said deficiency, or, for use in the supplementation of said at least one mineral and/or said at least one vitamin, in subjects in need.

Diseases, symptoms and/or disorders related to said insufficiency or deficiency of a mineral (such as magnesium, calcium, iron, zinc, iodine, selenium, copper or chromium) and/or of at least one vitamin (such as vitamin A, of group B (e.g. B12), C, D (e.g. D3) and/or E), which may be treated, in a preventive and/or curative and/or adjuvant manner, by administering said compositions or formulations of at least one mineral and/or of at least one vitamin of the present invention to a subject, may be selected from:
changes in the carbohydrate metabolism and/or diseases and disorders related thereto, such as for example, diabetes, preferably type II diabetes mellitus, hyperglycaemia, insulin resistance, high absorption of carbohydrates, deregulation of the blood glucose level and/or metabolic syndrome;
changes in the muscle energy metabolism and/or disorders related thereto, such as for example, decrease in muscle mass, decrease in muscle strength, decrease in physical resistance to muscle stress, poor absorption of amino acids;
dyslipidaemia or change in the lipid metabolism and/or diseases and disorders related thereto, such as for example, cholesterolaemia, high triglyceride levels, and obesity or overweight;
cognitive disorders or changes in the cognitive-emotional sphere;
cardiometabolic disorders: change in the immune system;
stress related to anxiety and depression, fatigue, chronic fatigue, and/or asthenia, in subjects in need.

The compositions and formulations comprising at least one mineral and/or at least one vitamin of the present invention may be administered to any category of subjects, including paediatric subjects, elderly subjects, sportsmen/sportswomen, pregnant women, preferably women both after pregnancy and women before, during and after the menstrual cycle.

In addition, forming an object of the present invention is said composition and formulation of at least one mineral of the invention (such as magnesium, calcium, iron, zinc, iodine, selenium, copper or chromium) and/or of at least one vitamin of the invention (such as, vitamin A, of group B (e.g. B12), C, D (e.g. D3) and/or E), according to any one of the described embodiments, for use in a method for the preventive and/or curative and/or adjuvant treatment (both therapeutic and non-therapeutic) to increase muscle energy metabolism (both for therapeutic purposes and non-therapeutic purposes) and, therefore, to increase muscle mass, increase muscle strength, increase physical resistance to muscle stress and reduce time to recover energy after a physical effort, in a subject in need or in a healthy subject; and/or to increase the physical or mental performance of a subject, preferably a healthy subject, for example a subject who does physical activity and/or studies.

Advantageously, the compositions or formulations based on iron (preferably iron (III) pyrophosphate) according to any one of the embodiments described in the present invention are used for preventing, reducing or treating sideropenia, anaemia and for increasing haemoglobin and ferritin values in subjects in need, such as for example women before, during and after pregnancy, during breast-feeding or during menstrual cycle, or in the elderly or in sportsmen/sportswomen, in change in the immune system. Said formulation is referred to as “cyclosome or cyclosomal iron”.

Advantageously, compositions or formulations based on magnesium (preferably magnesium oxide or magnesium hydroxide) according to any one of the embodiments described in the present invention are used for preventing, decreasing or treating musculoskeletal, cardiometabolic, emotional sphere (e.g. stress) and immune system disorders (e.g. physical and mental fatigue), in subjects in need. Said formulation is referred to as “magnesium cyclosomal or cyclosomal”.

Advantageously, the compositions or formulations based on calcium (preferably tricalcium phosphate) according to any one of the embodiments described in the present invention are used for preventing, decreasing or treating disorders related to pregnancy (i.e. developing the foetus), mood, bones, muscles and pressure, in subjects in need. Said formulation is referred to as “cyclosome or cyclosomal calcium”.

Advantageously, the compositions or formulations based on zinc (preferably zinc oxide) according to any one of the embodiments described in the present invention are used for preventing, reducing or treating disorders related to growth and development, metabolism (intermediary metabolism, DNA metabolism), immune system, vision and cognitive behaviour, in subjects in need. Said formulation is referred to as “cyclosome or cyclosomal zinc”.

Advantageously, the compositions or formulations based on selenium (preferably sodium (IV) selenite) according to any one of the embodiments described in the present invention are used for preventing, reducing or treating disorders related to pregnancy (i.e. development of the foetus), metabolic disorders and immune system disorders in subjects in need. Said formulation is referred to as “cyclosome or cyclosomal selenium”.

Advantageously, the compositions or formulations based on iodine (preferably sodium (V) iodate) according to any one of the embodiments described in the present invention are used for preventing, reducing or treating disorders related to pregnancy (i.e. Development of the foetus), mood, pressure, metabolism, cardiovascular disorders and energy deficiency disorders, in subjects in need. Said formulation is referred to as “cyclosome or cyclosomal iodine”.

Advantageously, the compositions or formulations based on chromium (preferably chromium (Ill) picolinate) according to any one of the embodiments described in the present invention are used for preventing, reducing or treating changes in the carbohydrate, lipid and energy metabolism, in subjects in need. Said formulation is referred to as “cyclosome or cyclosomal chromium”.

In order to treat a chromium deficiency (both for therapeutic purposes and for non-therapeutic or cosmetic purposes), and disease or symptoms related to said deficiency, the daily oral administration of a therapeutically effective amount known to the man skilled in the art of the chromium formulation of the present invention (cyclosomal chromium) or of the compositions thereof is recommended, for example an amount of chromium element (chromium (III) or chromium metal) comprised in the range from 10 μg to 500 μg (for example 50 μg, 100 μg, 150 μg, 200 μg, 250 μg, 300 μg, 350 μg, 400 μg, or 450 μg), preferably from 50 μg to 250 μg, more preferably from 150 μg to 250 μg, for example about 200-250 μg.

Advantageously, the compositions or formulations based on copper (preferably copper gluconate) according to any one of the embodiments described in the present invention are used for preventing, reducing or treating physical fatigue, anaemia and decreasing the number of white blood cells, osteoporosis, nerve lesions, tingling and loss of sensitivity to feet and hands, muscle weakness, connective tissue diseases (a group of autoimmune diseases characterised by inflammation of connective tissue), or disorders related to oxidative stress (oxidative stress is a mechanism involved in the pathogenesis of many neurodegenerative diseases such as alzheimer's, parkinson's and multiple sclerosis). Said formulation is referred to as “cyclosome or cyclosomal copper”.

In order to treat a copper deficiency (both for therapeutic purposes and for non-therapeutic or cosmetic purposes), and disease or symptoms related to said deficiency, the daily oral administration of a therapeutically effective amount known to the man skilled in the art of the copper formulation of the present invention (cyclosomal copper) or of the compositions thereof is recommended, for example an amount of copper element (copper (II) or copper metal) comprised in the range from 0.1 mg to 10 mg (for example 0.5 mg, 1 mg, 1.5 mg, 2 mg, 2.5 mg, 3 mg, 3.5 mg, 4 mg, 5 mg, 6 mg, 7 mg, 8 mg, or 9 mg) preferably from 1 mg to 4 mg, for example about 2 mg.

Advantageously, the compositions or formulations comprising vitamin B12 (according to any one of the embodiments described in the present invention) are used for preventing, reducing or treating a vitamin B12 deficiency. Diseases or symptoms related to a vitamin B12 deficiency are for example: weakness and physical fatigue, shortness of breath, tingling at the tips, memory loss or cognitive difficulties, difficulty in walking due to balance problems, hallucinations, anaemia, and pallor. Said formulation is referred to as “cyclosome or cyclosomal vitamin B12”

Advantageously, the compositions or formulations comprising vitamin C (according to any one of the embodiments described in the present invention) are used for preventing, reducing or treating a vitamin C deficiency. Diseases or symptoms related to vitamin C deficiency are for example: fatigue, depression, connective tissue disorders (for example collagen, gingivitis, petechiae, skin rashes, internal bleeding and delayed wound healing), brittleness of the nails, skin and hair following inflammation or medical treatment, and altered bone growth in children, change in the immune system, oxidative stress, decreased iron absorption. Said formulation is referred to as “cyclosome or cyclosomal vitamin C”

Advantageously, the compositions or formulations comprising vitamin D (according to any one of the embodiments described in the present invention) are used for preventing, reducing or treating a vitamin D deficiency. Diseases or symptoms related to vitamin D deficiency are for example: rickets in children and osteomalacia in adults (due to an alteration of bone mineralization), osteoporosis, and hyperparathyroidism, changes in the immune system. Said formulation is referred to as “cyclosome or cyclosomal vitamin D”.

Advantageously, the compositions or formulations comprising vitamin E (according to any one of the embodiments described in the present invention) are used for preventing, reducing or treating a vitamin E deficiency. Diseases or symptoms related to vitamin E deficiency (especially in paediatric subjects) are for example: a lack of reflexes and coordination, difficulty walking, muscle weakness, a form of anaemia in which red blood cells are destroyed (haemolytic anaemia), hair loss, skin diseases, weak immune system, fatigue, difficulty concentrating, vision problems, and loss of muscle tone, oxidative stress. Said formulation is referred to as “cyclosome or cyclosomal vitamin E”

In order to treat a vitamin C deficiency (both for therapeutic purposes and for non-therapeutic or cosmetic purposes), and disease or symptoms related to said deficiency, the daily oral administration of a therapeutically effective amount known to the man skilled in the art of the vitamin C formulation of the present invention (cyclosomal vitamin C) or of the compositions thereof is recommended, for example an amount of pure vitamin C (or vitamin c as such) comprised in the range from 100 mg to 1500 mg (for example, 200 mg, 400 mg, 600 mg, 800 mg, 1000 mg, or 1200 mg) preferably from 500 mg to 1000 mg, for example about 1000 mg (to date, the maximum daily dose allowed is 1000 mg).

In order to treat a vitamin B12 deficiency (both for therapeutic purposes and for non-therapeutic or cosmetic purposes), and disease or symptoms related to said deficiency, the daily oral administration of a therapeutically effective amount known to the man skilled in the art of the vitamin B12 formulation of the present invention (cyclosomal vitamin B12) or of the compositions thereof is recommended, for example at an amount of pure vitamin B12 (or vitamin B12 as such) comprised in the range from 0.5 μg to 1000 μg (for example, 1 μg, 1.5 μg, 2 μg, 2.5 μg, 5 μg, 10 μg, 50 μg, 100 μg, 200 μg, 300 μg, 400 μg, 500 μg, 600 μg, 700 μg, 800 μg, or 900 μg) preferably from 1 μg to 5 μg, for example about 2-2.5 μg (to date, the maximum daily dose allowed is 1000 μg).

In order to treat a vitamin E deficiency (both for therapeutic purposes and for non-therapeutic or cosmetic purposes), and disease or symptoms related to said deficiency, the daily oral administration of a therapeutically effective amount known to the man skilled in the art of the vitamin E formulation of the present invention (cyclosomal vitamin E) or of the compositions thereof is recommended, for example an amount of pure vitamin E (or vitamin E as such) comprised in the range from 0.5 mg to 80 mg (for example, 1 mg, 5 mg, 10 mg, 15 mg, 20 mg, 30 mg, 40 mg, 50 mg, 60 mg, or 70 mg) preferably from 5 mg to 15 mg, for example about 10 mg (to date, the maximum daily dose allowed is 60 mg).

In order to treat a vitamin D3 deficiency (both for therapeutic purposes and for non-therapeutic or cosmetic purposes), and disease or symptoms related to said deficiency, the daily oral administration of a therapeutically effective amount known to the man skilled in the art of the vitamin D3 formulation of the present invention (cyclosomal vitamin D3) or of the compositions thereof is recommended, for example an amount of pure vitamin D3 (or vitamin D3 as such) comprised in the range from 1 μg to 100 μg (for example, 5 μg, 10 μg, 15 μg, 20 μg, 25 μg, 30 μg, 50 μg, 100 μg, 150 μg, 200 μg, 250 μg, 300 μg, 350 μg, 400 μg, or 450 μg) preferably from 10 μg to 100 μg, for example about 25-50 μg.

The intake of the compositions or formulations of the present invention, comprising said at least one mineral and/or said at least one vitamin, shows significant changes in the subjects after the intake thereof (efficacy). In particular, the continuous intake of the compositions or formulations of the invention according to the doses described above considerably improves the relative levels of mineral/s and/or vitamins in the treated individuals (symptomatic or healthy individuals), both in the blood and in the organs, such as for example in the liver which is the organ where surplus iron is stored, in the form of Fe(III) and it carries a portion of iron (transport iron) through the blood to the tissues that need it.

Furthermore, the continued intake of the compositions or formulations of the present invention in the described doses does not have side effects and they may be taken by all types of subjects, including pregnant women, both on a full and on an empty stomach.

Lastly, the compositions or formulations of the present invention are easy to take, particularly if in solid form, and they do not have problems related to undesirable palatability.

In addition, the compositions or formulations of the present invention exhibit chemical/physical and organoleptic stability over time.

In the context of the present invention, the term “subject/s” is used to indicate human or animal subjects, preferably mammals (e.g. pets such as dogs, cats, horses, sheep or cattle). Preferably, the formulations and compositions of the invention are for use in treatment methods on human subjects.

The compositions of the invention or the formulations of at least one mineral and/or of at least one vitamin of the invention (cyclosome or cyclosomal mineral and/or vitamin) may be administered for a period of time comprised in the range from 3 days to 60 days or 90 days.

Forming an object of the present invention is a method for the preventive and/or curative and/or adjuvant treatment of an insufficiency or deficiency of a mineral (such as magnesium, calcium, iron, zinc, iodine, selenium, copper and/or chromium) and/or at least one vitamin (such as vitamin A, of group B (e.g. B12), C, D (e.g. D3) and/or E) or a disease, symptom and/or disorder related to said deficiency, wherein said method provides for the administration of an effective amount (therapeutically effective amount) of the composition of the invention or of the formulation of at least one mineral and/or at least one vitamin of the invention to a subject in need.

The term “therapeutically effective amount” is used to indicate the amount of formulation or compound that elicits the biological or medicinal response in a tissue, system or subject that is sought and defined by a man skilled in the art.

Furthermore, forming an object of the present invention is the use (non-therapeutic) of the compositions and formulations of at least one mineral of the invention (such as, magnesium, calcium, iron, zinc, iodine, selenium, copper and/or chromium) and/or of at least one vitamin (such as, vitamin A, of group B (e.g. B12), C, D (e.g. D3) and/or E) according to any one of the described embodiments, to increase muscle energy metabolism and, therefore, to increase muscle mass, to increase muscle strength, to increase physical resistance to muscle stress and to reduce time to recover energy after a physical effort, in a healthy subject; and/or to increase the physical or mental performance of a healthy subject; and/or to support or strengthen immune defences in a healthy subject.

Forming an object of the present invention is a process (in brief, the process of the invention) for the preparation of the solid form formulations of at least one mineral of the invention (such as magnesium, calcium, iron, zinc, iodine, selenium, copper or chromium) (cyclosome or cyclosomal mineral) and/or for the preparation of the solid form formulations of at least one vitamin (such as vitamin A, of group B (e.g. B12), C, D (e.g. D3) and/or E) (cyclosome or cyclosomal vitaminl).

In a first embodiment of the invention, said process of the invention (in short, first process of the invention) is described in patent document WO 2014/009806 A1 from page 7 line 1 to page 8 line 20, incorporated in the present application for reference.

In a second embodiment of the invention, said process of the invention (in short, second process of the invention) is described in patent document WO 2014/009806 A1 from page 8 line 22 to page 10 line 21, incorporated in the present application for reference.

According to the present invention, said first process of the invention and said second process of the invention are applied as described in patent document WO 2014/009806 A1 considering that (c-i), (c-ii) and (c-iii) can be used in substitution or in addition to (d) and at the amounts suitable to obtain the formulations of the present invention, and that the term (b) lecithin is to be read as representative of the phospholipid class (wherein (c-i) is carrageenan, (c-ii) is acacia gum, (c-iii) is fucoidan, and (d) is sucrester).

Thus, in the context of the present invention, the term “cyclosome or cyclosomal mineral” indicates a formulation of said at least one mineral that can be obtained by processing a mineral salt or complex or oxide (a) together with a phospholipid or phosphatidylcholine or lecithin (b), a first agent (c) such as a carrageenan (c-i) or a acacia gum (c-ii) or a fucoidan (c-iii) and, optionally, a sucrester (d) and/or a starch (e), according to said first method or said second method, described in WO 2014/009806 A1, or as described in the present invention.

In the context of the present invention, the term “cyclosome or cyclosomal vitamin” indicates a formulation of said at least one vitamin that can be obtained by processing a vitamin (a) together with a phospholipid or phosphatidylcholine or lecithin (b), a first agent (c) (such as a carrageenan (c-i) or a acacia gum (c-ii)), and, optionally, a sucrester (d) and/or a starch (e), according to said first process or said second process, described in WO 2014/009806 A1, or as described in the present description.

Said process according to the invention is carried out by mixing the components (a), (b), (c) and, optionally, (d) and/or (e) according to the percentages by weight defined in the context of the present invention in order to obtain the formulations according to the invention comprising at least one mineral and/or at least one vitamin, preferably formulations according to the invention comprising a mineral or a vitamin.

Preferably, said process of the invention for the preparation of a formulation according to the invention comprising a mineral and/or a vitamin (preferably, only a mineral or only a vitamin), for example according to an embodiment described in WO 2014/009806 A1, comprises the steps of:
step 1: preparing a mineral (salt, oxide or complex of the mineral) or a vitamin in the form of powder or granules (preferably either only a mineral or only a vitamin);
step 2: mixing said mineral or vitamin with a phospholipid (b), preferably lecithin (for example sunflower lecithin) to obtain a mixture of step 2; advantageously said lecithin is not a hydrolysed or enzymatically hydrolysed lecithin;
step 3: mixing said mixture of step 2 with a polysaccharide (c) (e.g. (c-i) carrageenan or acacia gum) and, optionally, with a sucrester (d), to obtain a mixture of step 3;
step 4 (optional): mixing said mixture of step 3 with a gelatinised or pre-gelatinised starch of plant origin (e), preferably pre-gelatinised, for example pre-gelatinised rice starch;
step 5 (optional after step 3 and/or after step 4): sieve, preferably with at least one sieve having a size (or nominal sieve opening) comprised in the range from 150 μm to 1400 μm (for example 180 μm, 212 μm, 250 μm, 300 μm, 355 μm, 425 μm, 500 μm, 600 μm, 710 μm, 850 μm, 1000 μm, or 1180 μm), preferably from 600 μm to 850 μm, for example 710 μm (25 US Mesh).

Advantageously, said mixing steps 2, 3 and/or 4 are carried out at room temperature (from 15° C. to 30° C., preferably at about 25° C.) for a period of time comprised from 10 minutes to 120 minutes, preferably from 15 minutes to 60 minutes, for example about 30 minutes.

Advantageously, said mixing steps 2, 3 and/or 4 are carried out in the absence of solvent (dry mixing).

In the embodiment in which said sucrester (d) is present in the formulation of the invention, said step 3 of the process of the invention may provide for mixing said mixture of step 2 first with the polysaccharide and then with the sucrester, or, alternatively, mixing said mixture of step 2 first with the sucrester and then with the polysaccharide, or, alternatively and preferably, mixing said mixture of step 2 simultaneously with the sucrester and the polysaccharide.

Said process for the preparation of the formulation of the invention (based on mineral or vitamin) may provide for that step 2 and step 3 be carried out simultaneously, i.e. the mineral or vitamin are mixed with the phospholipid, the polysaccharide and, optionally, the sucrester in a single step (step 2+3).

According to an example of the process of the invention for the preparation of a formulation comprising a mineral or a vitamin, said process does not comprise a spray-dry step.

Forming an object of the present invention is to provide a further process for the preparation of a composition (in short, composition process of the invention) comprising at least one formulation comprising a mineral according to the present invention (such as magnesium, calcium, iron, zinc, iodine, selenium, copper or chromium) (cyclosomal mineral) and/or at least one formulation comprising a vitamin according to the present invention (such as vitamin A, of group B (e.g. B12), C, D (e.g. D3) and/or E) (cyclosomal vitamin).

Said composition process of the invention comprises the following steps:
step (i) of preparing (or producing) at least one solid form formulation of a mineral according to the present invention (such as magnesium, calcium, iron, zinc, iodine, selenium, copper or chromium) (cyclosomal mineral) and/or preparing (or producing) at least one solid form formulation of a vitamin according to the present invention (such as, vitamin A, of group B (e.g. B12), C, D (e.g. D3) and/or E) (cyclosomal vitamin), to obtain at least one formulation based on a mineral and/or at least one formulation based on a vitamin (in short, at least one formulation step (i));
optional step (ii) of mixing said at least one (or two or three) formulation comprising a mineral according to the invention (obtained from step (i)) and said at least one (or two or three) formulation comprising a vitamin according to the invention (obtained from step (i)), or, alternatively, mixing at least two (or three or four) formulations each comprising a mineral according to the invention, or, alternatively, mixing at least two (or three or four) formulations each comprising a vitamin according to the invention, to obtain a mixture of step (ii);
step (iii) of mixing said at least one formulation of step (i) or said mixture of step (ii) with at least one additive and/or excipient to obtain a composition according to the invention comprising at least one mineral and/or at least one vitamin (each in cyclosomal form) and at least one additive/excipient; and
optional sieving step (iv), after step (ii) and/or after step (iii), preferably with at least one sieve having a size (or nominal sieve opening) comprised in the range from 150 μm to 1400 μm (for example 180 μm, 212 μm, 250 μm, 300 μm, 355 μm, 425 μm, 500 μm, 600 μm, 710 μm, 850 μm, 1000 μm, or 1180 μm), preferably from 600 μm to 850 μm, for example 710 μm (25 US Mesh).

Advantageously, said mixing step (ii) and/or step (iii) are carried out at room temperature (from 15° C. to 30° C., preferably at about 25° C.) for a period of time comprised from 10 minutes to 120 minutes, preferably from 15 minutes to 60 minutes, for example about 30 minutes.

Said mixing step (ii) may provide for mixing at least one mineral and at least one vitamin, or mixing several minerals (for example two, three, four, or five minerals) without vitamins, or mixing several vitamins (for example two, three, four, or five vitamins) without minerals. Thus, a composition that can be obtained according to said composition process of the invention may comprise a mineral and a vitamin, or a mineral and several vitamins (for example two, three, four, or five vitamins), or several minerals (for example two, three, four, or five minerals) and a vitamin, or several minerals (for example, two, three, four, or five minerals) and several vitamins (for example, two, three, four, or five vitamins), or several minerals (for example, two, three, four, or five minerals) without vitamins, or several vitamins (for example, two, three, four, or five vitamins) without minerals.

Unless otherwise specified, the term composition or other comprising a component at an amount “comprised in a range from x to y” is used to indicate that said component may be present in the composition or formulation or other at all amounts present in said range, even if not explicitly stated, range extremes comprised.

Unless otherwise specified, the content of a component in a composition or formulation refers to the percentage by weight of that component based on the total weight of the composition or formulation.

Unless specified otherwise, the indication that a composition “comprises” one or more components means that other components—besides the one, or the ones, indicated specifically—can be present and the indication that a composition “consists” of determined components means that the presence of other components is excluded.

Examples of embodiments (FRa-nr) of the present invention are reported below:

FRa-1. A formulation in solid form comprising, or alternatively, consisting of:
(a) at least one mineral in form of a salt or complex of said mineral, wherein said mineral is selected from among the group comprising or, alternatively, consisting of: (a-i) magnesium, (a-ii) calcium, (a-iii) iron, (a-iv) zinc, (a-v) iodine, (a-vi) selenium, (a-vii) chromium, (a-viii) copper (II) and mixtures thereof;
(b) at least one phospholipid;
(c) at least one first agent selected from the group comprising or, alternatively, consisting of:

(c-i) at least one carrageenan,

(c-ii) at least one acacia gum,

(c-iii) at least one fucoidan, and mixtures thereof.

FRa-2. The formulation according to FRa-1, wherein said (c) at least one first agent consists of said (c-i) at least one carrageenan; preferably carrageenan E407.

FRa-3. The formulation according to FRa-1, wherein said (c) at least one first agent consists of said (c-ii) at least one acacia gum; preferably an acacia gum E414; more preferably an acacia gum E414 having an average molecular weight comprised in the range from 250,000 to 400,000, more preferably an average molecular weight of 350,000.

FRa-4. The formulation according to FRa-1, wherein said (c) at least one first agent consists of said (c-iii) at least one fucoidan; preferably a fucoidan having an average molecular weight comprised in the range from 20,000 to 30,000.

FRa-5. The formulation according to any one of Fra-1-Fra-4, wherein said formulation further comprises (d) at least one sucrester or a fatty acid carbohydrate ester; preferably sucrester E473; more preferably sucrester E473 comprising from 70% to 90% by weight, with respect to the total weight of the sucrester, of monoesters obtained through the esterification of the sucrose with one or more fatty acids of plant origin, preferably said fatty acids are selected from stearic acid and/or palmitic acid.

FRa-6. The formulation according to any one of FRa-1-FRa-5, wherein said formulation further comprises (e) at least one gelatinised or pre-gelatinised starch of plant origin; preferably, wherein said (e) at least one starch of plant origin is selected from rice starch and/or corn starch; preferably, wherein said (e) is pre-gelatinised rice starch.

FRa-7. The formulation according to any one of Fra-1-Fra-6, wherein said (b) phospholipid is a phosphoglyceride; preferably a phosphatidylcholine or lecithin; more preferably, wherein said (b) phospholipid is a lecithin E322 selected from sunflower lecithin, corn lecithin, soy lecithin and mixtures thereof.

FRa-8. The formulation according to any one of Fra-1-Fra-7, wherein
said (b) phospholipid, preferably lecithin E322, more preferably solid sunflower lecithin E322, is comprised in the range from 0.05% to 15%, preferably from 0.1% to 5%, more preferably from 0.1% to 2;
said (c) first agent, selected from among the group comprising or, alternatively, consisting of: (c-i) carrageenan, (c-ii) acacia gum, (c-iii) fucoidan and a mixture thereof, is comprised in a % comprised in the range from 1% to 50%, preferably from 1% to 35%, more preferably from 1% to 20%; and, optionally,
said (d) sucrester, preferably sucrester E473, is comprised in a % comprised in the range from 1% to 50%, preferably from 1% to 35%, more preferably from 1% to 20%;

wherein said % are % by weight with respect to the total weight of the formulation.

FRa-9. A composition comprising: the solid form formulation of said at least one mineral according to any one of FRa-1-FRa-8; and, optionally, at least one acceptable pharmaceutical or food grade additive and/or excipient.

FRa-10. The composition according to the composition FRa-9 for use in a method for the preventive and/or curative and/or adjuvant treatment of a deficiency of said (a) at least one mineral, and of diseases, symptoms or disorders related to or deriving from said deficiency, in a subject in need.

Further examples of embodiments (FRb-nr) of the present invention are reported below:

FRb-1. A formulation in solid form comprising, or alternatively, consisting of:
(a) a nutrient, wherein said nutrient is a mineral or a vitamin,

wherein said mineral is selected from the group comprising or, alternatively, consisting of: (a-i) magnesium, (a-ii) calcium, (a-iii) iron, (a-iv) zinc, (a-v) iodine, (a-vi) selenium, (a-vii) chromium, and (a-viii) copper (II), and wherein said mineral is in the form of a salt or complex or oxide of said mineral; and

wherein said vitamin is selected from the group comprising or, alternatively, consisting of: vitamin B12, vitamin C, vitamin D3, vitamin E;
(b) a phospholipid;
(c) a first agent selected from (c-i) a carrageenan or (c-ii) an acacia gum.

FRb-2. The formulation according to FRb-1, wherein said formulation further comprises (d) at least one sucrester or fatty acid carbohydrate ester;

preferably sucrester E473; more preferably sucrester (E473) comprising from 70% to 90% by weight, with respect to the total weight of the sucrester, of monoesters obtained through the esterification of sucrose with one or more fatty acids of plant origin, preferably said fatty acids are selected from stearic acid and/or palmitic acid.

FRb-3. The formulation according to FRb-1 or Frb-2, wherein said (c) first agent consists of said (c-i) carrageenan; preferably carrageenan E407.

FRb-4. The formulation according to FRb-1 or FRb-2, wherein said first agent (c) consists of said (c-ii) acacia gum;

preferably an acacia gum E414; more preferably an acacia gum E414 having an average molecular weight comprised in the range from 250,000 to 400,000.

FRb-5. The formulation according to any one of FRb-1-FRb-4, wherein said formulation further comprises (e) a gelatinised or pre-gelatinised starch of plant origin;

preferably, wherein said (e) starch is selected from rice starch or corn starch; preferably, wherein said starch is pre-gelatinised rice starch.

FRb-6. The formulation according to any one of FRb-1-FRb-5, where said (b) phospholipid is a phosphatidylcholine or lecithin; preferably wherein said (b) phospholipid is a lecithin (E322) selected from the group comprising or, alternatively, consisting of: sunflower lecithin, corn lecithin, soy lecithin and mixtures thereof.

FRb-7. The formulation according to any one of FRb-1-FRb-6, wherein a weight ratio between said first agent (c) and said phospholipid (b) [(c):(b)], or, alternatively, a weight ratio between the weight of the sum of said first agent (c) and said sucrester (d), and the weight of said phospholipid [[(c)+(d)]:(b)] is comprised from 50:1 to 10:1, preferably from 40:1 to 10:1, more preferably from 30:1 to 15:1; preferably wherein said (b) phospholipid is a lecithin.

FRb-8. The formulation according to any one of FRb-2-FRb-7, wherein on 100 parts by weight of a sum of said first agent (c) and said sucrester (d), said (c) first agent is comprised in percentage by weight from 1% to 100% and said (d) sucrester is comprised from 0% (absent) to 99%;

preferably said (c) first agent from 50% to 95% and said (d) sucrester from 5% to 50%;

more preferably, said (c) first agent from 70% to 80% and said (d) sucrester from 20% to 30%.

FRb-9. A composition comprising:
at least one solid form formulation of said nutrient according to any one of FRb-1-FRb-8; and
at least one acceptable pharmaceutical or food grade additive and/or excipient.

FRb-10. The composition according to FRb-9, wherein said nutrient is a mineral.

FRb-11. The composition according to FRb-9, wherein said nutrient is a vitamin.

FRb-12. The composition according to FRb-9, wherein said composition comprises:
at least one solid form formulation of mineral according to any one of FRb-1-FRb-8;
at least one solid form formulation of a vitamin according to any one of FRb-1-FRb-8; and at least one acceptable pharmaceutical or food grade additive and/or excipient.

FRb-13. The formulation or the composition according to any one of FRb-1-FRb-12 for use as medicament.

FRb-14. The formulation or the composition according to any one of FRb-1-FRb-13 for use in a method for the preventive and/or curative and/or adjuvant treatment of a deficiency of said (a) mineral and/or vitamin, and of diseases, symptoms or disorders related to or deriving from said deficiency, in a subject in need.

FRb-15. The formulation or the composition for use according to FRB-14, wherein said diseases or symptoms related to or deriving from said deficiency are selected from the group comprising or, alternatively, consisting of:
sideropenia, anaemia, poor iron absorption, haemolytic anaemia;
changes in carbohydrate metabolism and related diseases and disorders, diabetes, type II diabetes mellitus, hyperglycaemia, insulin resistance, high absorption of carbohydrates, deregulation of blood glucose level, metabolic syndrome;
changes in the muscle energy metabolism and/or disorders related thereto decrease in muscle mass, decrease in muscle strength, decrease in physical resistance to muscle stress, poor absorption of amino acids;
dyslipidaemia or change in the lipid metabolism and diseases and disorders related thereto, cholesterolaemia, high triglyceride levels, and obesity or overweight;
cognitive disorders or changes in the cognitive-emotional sphere;
cardiometabolic disorders:
change in the immune system;
stress related to anxiety and depression, fatigue, chronic fatigue, asthenia, a lack of reflexes and coordination;
rickets or delayed bone growth in children, osteomalacia in adults, osteoporosis, and hyperparathyreodism.
gingivitis; brittleness of the nails, hair and/or skin following inflammations or medical treatment.

FRb-16. Non-therapeutic use of the formulation or composition according to any one of the FRb-1-FRb-12 for the supplementation of said (a) mineral and/or vitamin in a healthy subject.

FRb-17. Non-therapeutic use according to FRb-16, where said use is selected from: increasing muscle mass, increasing muscle strength, increasing physical resistance to muscular effort, reducing time to recover energy after a physical effort, increasing mental efficiency, strengthening nails and hair.

FRb-18. A process for the preparation of the formulation according to any one of FRb-1-FRb-8 comprising the steps of:
(1) providing a mineral or a vitamin (a) in the form of powder or granules, wherein said mineral is in the form of a salt, oxide or complex of said mineral, to obtain a mineral or a vitamin of step 1;
(2) mixing—in the absence of solvent—said mineral or said vitamin of step 1 with a phospholipid (b), preferably lecithin, to obtain a mixture of step 2;
(3) mixing—in the absence of solvent—said mixture of step 2 with a polysaccharide (c) selected from (c-i) carrageenan or (c-ii) acacia gum and, optionally, with a sucrester (d), to obtain a mixture of step 3 or the formulation;
(4), optionally, mixing said mixture of step 3 with a gelatinised or pre-gelatinised starch of plant origin (e), preferably pre-gelatinised rice starch, to obtain the formulation.

FRb-19. A process for the preparation of the composition according to claim 12 comprising the steps of:
(i) preparing at least one solid form formulation of a mineral and preparing at least one solid form formulation of a vitamin according to any one of claims 1 to 8 or, alternatively, producing at least one solid form formulation of a mineral and producing at least one solid form formulation of a vitamin according to FRb-18 to obtain said at least one solid form formulation of a mineral and said at least one solid form formulation of a vitamin;
(ii) mixing said at least one solid form formulation of a mineral and said at least one solid form formulation of a vitamin to obtain a mixture of step (ii).
(iii) mixing said mixture of step (ii) with at least one additive and/or excipient to obtain the composition.

### EXAMPLES of FORMULATIONS ACCORDING TO THE INVENTION

### Example 1

A solid formulation (about 100 mg) according to the present invention based on iron consisting of iron pyrophosphate from about 40 mg to 50 mg (Fe(3+) about 10-12 mg), lecithin (for example, non-hydrolysed sunflower lecithin) from 0.3 mg to 2 mg, carrageenan or acacia gum from 15 mg to 20 mg, pre-gelatinised rice starch from 30 mg to 45 mg (sucrester absent).

### Example 2

A solid formulation (about 100 mg) according to the present invention based on iron consisting of: iron pyrophosphate from about 40 mg to 50 mg (Fe(3+) about 10-12 mg), lecithin (for example, non-hydrolysed sunflower lecithin) from 0.3 mg to 2 mg, carrageenan or acacia gum from 2.5 mg to 5 mg, sucrester from 10 mg to 20 mg, pre-gelatinised rice starch from 30 mg to 45 mg (polysaccharide:sucrester weight ratio about 25 75).

### Example 3

A solid formulation (about 100 mg) according to the present invention based on iron consisting of: iron pyrophosphate from about 40 mg to 50 mg (Fe(3+) about 10-12 mg), lecithin (for example, non-hydrolysed sunflower lecithin) from 0.3 mg to 2 mg, carrageenan or acacia gum from 10 mg to 20 mg, sucrester from 2.5 mg to 5 mg, pre-gelatinised rice starch from 30 mg to 45 mg (polysaccharide sucrester weight ratio about 75 25).

### Example 4

A solid formulation (about 100 mg) according to the present invention based on magnesium consisting of: magnesium oxide from about 45 mg to 60 mg (Mg(2+) about 32-38 mg), lecithin (for example, non-hydrolysed sunflower lecithin) from 0.5 mg to 2 mg, polysaccharide (i.e. carrageenan or acacia gum) and sucrester from 15 mg to 20 mg (polysaccharide:sucrester weight ratio about 75:25 or 50:50 or 25:75), pre-gelatinised rice starch from 20 mg to 35 mg.

### Example 5

A solid formulation (about 100 mg) according to the present invention based on zinc consisting of: zinc oxide from about 45 mg to 60 mg (zinc element about 40-50 mg), lecithin (for example, non-hydrolysed sunflower lecithin) from 0.5 mg to 2 mg, polysaccharide (i.e. carrageenan or acacia gum) and sucrester from 15 mg to 20 mg (polysaccharide sucrester weight ratio about 75:25 or 50:50 or 25:75), pre-gelatinised rice starch from 20 mg to 35 mg.

### Example 6

A solid formulation (about 100 mg) according to the present invention based on iodine consisting of: sodium iodate from about 1 mg to 3 mg (iodine element about 0.5-1.5 mg), lecithin (for example, non-hydrolysed sunflower lecithin) from 0.5 mg to 2 mg, polysaccharide (i.e. carrageenan or acacia gum) and sucrester from 15 mg to 20 mg (polysaccharide:sucrester weight ratio about 75:25 or 50:50 or 25:75), pre-gelatinised rice starch from 75 mg to 85 mg.

### Example 7

A solid formulation (about 100 mg) according to the present invention based on calcium consisting of: tricalcium phosphate from about 90 mg to 99 mg (Ca(2+) about 31-37 mg), lecithin (for example, non-hydrolysed sunflower lecithin) from 0.5 mg to 2 mg, polysaccharide (i.e. carrageenan or acacia gum) and sucrester from 1 mg to 4 mg (polysaccharide:sucrester weight ratio about 75:25 or 50:50 or 25:75), starch absent or present in a low percentage (for example 0.5-5% w/w).

### Example 8

A solid formulation (about 100 mg) according to the present invention based on selenium consisting of: sodium selenite from about 1 mg to 4 mg (selenium element about 0.5-2 mg, for example about 1 mg), lecithin (for example, non-hydrolysed sunflower lecithin) from 0.5 mg to 2 mg, polysaccharide (i.e. carrageenan or acacia gum) and sucrester from 15 mg to 20 mg (polysaccharide sucrester weight ratio about 75:25 or 50:50 or 25:75), pre-gelatinised rice starch from 75 mg to 85 mg.

### Example 9

A solid formulation (about 100 mg) according to the present invention based on: chromium picolinate from about 70 mg to 80 mg (Cr(3+) about 8-10 mg), lecithin (for example, non-hydrolysed sunflower lecithin) from 0.3 mg to 1 mg, polysaccharide (i.e. carrageenan or acacia gum) and sucrester from 15 mg to 20 mg (polysaccharide sucrester weight ratio about 75:25 or 50:50 or 25:75), pre-gelatinised rice starch from 5 mg to 15 mg.

### Example 10

A solid formulation (about 30-40 mg) according to the present invention based on: copper gluconate from about 13.5 mg to 18 mg (Cu(2+) about 2-2.5 mg), lecithin (for example, non-hydrolysed sunflower lecithin) from 0.17 mg to 0.23 mg, polysaccharide (i.e. carrageenan or acacia gum) and sucrester from 5 mg to 7 mg (polysaccharide:sucrester weight ratio about 75:25 or 50:50 or 25:75), pre-gelatinised rice starch from 11 mg to 15 mg.

### Example 11

A composition according to the invention comprising:

(A) a solid formulation (about 250-300 mg, for example about 270 mg) according to the present invention based on iron consisting of: iron pyrophosphate from about 110 mg to 135 mg (Fe(3+) 27-33 mg, for example about 30 mg), lecithin (for example, non-hydrolysed sunflower lecithin) from 1.5 mg to 1.8 mg (for example about 1.6), polysaccharide (carrageenan or acacia gum) and sucrester from 40 mg to 50 mg (for example about 45 mg) (polysaccharide:sucrester weight ratio about 75:25 or 50:50 or 25:75), pre-gelatinised rice starch from 95 mg to 115 mg (for example about 100 mg); and

(B) a solid formulation (10-15 mg, for example about 12 mg)) according to the present invention based on vitamin D3 consisting of: commercial vitamin D3 from about 8.5 mg to 12.5 mg (pure vitamin D3 intake from 20 μg to 30 μg (about 0.20%), lecithin (for example, non-hydrolysed sunflower lecithin) from 0.7 mg to 0.10 mg, polysaccharide (i.e. carrageenan or acacia gum) and sucrester from 1.8 mg to 2 mg (polysaccharide:sucrester weight ratio about 75:25 or 50:50 or 25:75), pre-gelatinised rice starch from 0.05 mg to 0.25 mg.

### Example 12

A composition according to the invention comprising:

(A) a solid formulation (30-40 mg, for example about 35 mg) according to the present invention based on zinc consisting of: zinc oxide from about 15 mg a 22 mg (Zn(2+) about 12-18 mg, for example 15 mg), lecithin (for example, non-hydrolysed sunflower lecithin) from 0.3 mg to 0.4 mg (for example about 0.35 mg), polysaccharide (i.e. carrageenan or acacia gum) and sucrester from 5 mg to 8 mg (for example about 6 mg) (polysaccharide:sucrester weight ratio about 75:25 or 50:50 or 25:75), pre-gelatinised rice starch from 8.5 mg to 12 mg (for example about 10 mg); and

(B) a solid formulation (20-30 mg, for example about 24 mg) according to the present invention based on vitamin D3 consisting of: commercial vitamin D3 from about 17 mg to 25 mg (pure vitamin D3 intake from 40 μg to 60 μg (for example about 50 μg), lecithin (for example, non-hydrolysed sunflower lecithin) from 0.14 mg to 0.20 mg (for example about 0.17 mg), polysaccharide (i.e. carrageenan or acacia gum) and sucrester from 2.5 mg to 4 mg (for example about 3 mg) (polysaccharide:sucrester weight ratio about 75:25 or 50:50 or 25:75), pre-gelatinised rice starch from 0.1 mg to 0.5 mg; and

(C) a solid formulation (1-3 g, for example about 2 g) according to the present invention based on vitamin C consisting of: vitamin C from 0.5 g to 2 g (for example about 1 g), lecithin (for example, non-hydrolysed sunflower lecithin) from 0.01 g to 0.03 g, polysaccharide (i.e. carrageenan or acacia gum) and sucrester from 0.15 g to 0.3 g (polysaccharide:sucrester weight ratio about 75:25 or 50:50 or 25:75), pre-gelatinised rice starch from 0.3 g to 0.6 g.

### Example 13

A composition according to the invention comprising:

(A) a solid formulation (about 2.2 mg) according to the present invention based on chromium consisting of: chromium picolinate from about 1 mg to 2 mg (for example about 1.65 mg) (equivalent to 0.12-0.24 mg Cr(3+); for example 0.20 mg), lecithin (for example, non-hydrolysed sunflower lecithin) from 0.01 mg to 0.02 mg (for example 0.013), (carrageenan or acacia gum) and sucrester from 0.3 mg to 0.45 mg (for example about 0.38 mg) (polysaccharide:sucrester weight ratio about 75: 25 or 50:50 or 25:75), pre-gelatinised rice starch from 0.1 mg to 0.25 mg (for example about 0.18 mg); and

(B) a solid formulation (5 μg) according to the present invention based on vitamin B12 consisting of: Vitamin B12 from 2 μg to 3 μg (for example about 2.5 μg), lecithin (for example, non-hydrolysed sunflower lecithin) from 0.025 μg to 0.075 μg; polysaccharide (i.e. carrageenan or acacia gum) and sucrester from 0.70 μg to 1 μg (polysaccharide:sucrester weight ratio about 75:25 or 50:50 or 25:75), pre-gelatinised rice starch from 1.5 μg to 2 μg.

### Example 14

A solid formulation (1-3 g, for example about 2 g) according to the present invention based on vitamin C (sucrosomial® vitamin C) consisting of: vitamin C from 0.5 g to 2 g (for example about 1 g), lecithin (for example, non-hydrolysed sunflower lecithin) from 0.01 g to 0.03 g, polysaccharide (i.e. carrageenan or acacia gum) and sucrester from 0.15 g to 0.3 g (polysaccharide:sucrester weight ratio about 75: 25 or 50:50 or 25:75), and pre-gelatinised rice starch from 0.3 g to 0.6 g.

### Example 15

A solid formulation (2-6 mg, for example about 4 mg) according to the present invention based on vitamin B12 (sucrosomial® vitamin B12) consisting of: vitamin B12 from 1 mg to 3 mg (for example about 2 mg), lecithin (for example, non-hydrolysed sunflower lecithin) from 0.02 g to 0.06 g, polysaccharide (i.e. carrageenan or acacia gum) and sucrester from 0.3 g to 0.6 g (polysaccharide:sucrester weight ratio about 75:25 or 50:50 or 25:75), and pre-gelatinised rice starch from 0.6 g to 1.2 g.

### Example 16

A solid formulation (10-30 mg, for example about 20 mg) according to the present invention based on vitamin E (sucrosomial® vitamin E) consisting of: vitamin E from 5 mg to 15 mg (for example about 10 mg), lecithin (for example, non-hydrolysed sunflower lecithin) from 0.1 mg a 0.3 mg, polysaccharide (i.e. carrageenan or acacia gum) and sucrester from 1.7 g to 5 mg (polysaccharide:sucrester weight ratio about 75:25 or 50:50 or 25:75), and pre-gelatinised rice starch from 3 mg to 9 mg.

### Example 17

A solid formulation (20-30 mg, for example about 24 mg) according to the present invention based on vitamin D3 (sucrosomial® vitamina D3) consisting of: commercial vitamin D3 from about 17 mg to 25 mg (pure vitamin D3 intake from 40 μg to 60 μg (for example about 50 μg), lecithin (for example, non-hydrolysed sunflower lecithin) from 0.14 mg to 0.20 mg (for example about 0.17 mg), polysaccharide (i.e. carrageenan or acacia gum) and sucrester from 2.5 mg to 4 mg (for example about 3 mg) (polysaccharide:sucrester weight ratio about 75:25 or 50:50 or 25:75), and pre-gelatinised rice starch from 0.1 mg to 0.5 mg.

Experimental Part

I. Study of Permeation of Fe³⁺ Through Isolated Rat Intestine

I.1. Method

For permeation studies, the intestinal mucosa was isolated from male Wistar rats weighing 250-300 g. After sacrificing the rat, the first 20 cm of the jejunum was immediately removed. The isolated intestine was cut into 1.5 cm strips, the lumen content was gently removed and mounted in a Ussing-type cell (0.78 cm² exposed surface) without removing the underlying muscle layer. 1 ml of phosphate buffer pH 6.8, 0.13 M, made isotonic by adding sodium chloride (PBS 6.8) was added to the apical side and 3 ml of a pH 7.4, 0.13 M, isotonic buffer solution (PB 7.4) was added to the basolateral side (acceptor medium). To ensure oxygenation and agitation, a mixture of 95% O₂ and 5% CO₂ was bubbled in each compartment. Ussing chambers were placed in a water bath at 37° C.

After 20 minutes of equilibration, the medium in each donor compartment was replaced with 1 ml of the suspension of the composition to be tested which had been previously treated to simulate its transit in the stomach. 0.2 g of pepsin were solubilised in 5 ml of an aqueous solution of HCI 0.1 M so as to simulate gastric digestion. 2.5 g of Chelex-100 resin were added thereto and they were stirred 30 minutes. The suspension was then transferred to the column and once the eluate was collected, a further 5 ml of 0.1 M Hct were added to the column. The final volume of the eluate was 8 ml. A predetermined amount of sample was placed in a container with a screw cap, then 10 ml of an aqueous solution containing NaCl (140 mM) and KCl (5 mM) were added, the suspension was brought to pH 2 using HCI 5 M and 0.5 ml of the pepsin solution were added thereto. The suspension was then degassed with carboxicarb so as to create the anaerobic conditions present in the gastrointestinal tract, then the well-closed container was put in a shaker bath at 37° C. for 60 minutes. As a control, experiments similar to the one described were carried out while maintaining PBS pH 6.8 in the donor compartment.

1 ml of sample was collected from the receiving compartment at 30-minute intervals for a total of 240 minutes and replaced with the same volume of fresh medium. The amount of Fe³⁺ permeated was determined according to an analytical method.

I.2. Composition Under Analysis

The compositions under analysis all contained the same iron concentration (1.34 mg/mL):
Iron (III) pyrophosphate as such (embodiment not according to the invention).
Fe-Suc-V₀₀₀: comprising Fe(III) pyrophosphate, sunflower lecithin, sucrester and pre-gelatinised rice starch; does not include carrageenan, acacia gum and fucoidan (embodiment not according to the invention, reference composition).
Fe-Car25-V₀₀₁ (Table 1): comprising Fe(III) pyrophosphate, sunflower lecithin, carrageenan (25% w/w) and sucrester (75% w/w), and pre-gelatinised rice starch (embodiment according to the invention).
Fe-Car75-V₀₀₁ (Table 2): comprising Fe(III) pyrophosphate, sunflower lecithin, carrageenan (75% w/w) and sucrester (25% w/w), and pre-gelatinised rice starch (embodiment according to the invention).
Fe-GA75-V₀₀₁ (Table 3): comprising Fe(III) pyrophosphate, sunflower lecithin, gum arabic (75% w/w) and sucrester (25% w/w), and pre-gelatinised rice starch (embodiment according to the invention).
Fe-GA95-V₀₀₁: comprising Fe(III) pyrophosphate, sunflower lecithin, gum arabic (95% w/w) and sucrester (5% w/w), and pre-gelatinised rice starch (embodiment according to the invention).
Fe-GA100-V₀₀₁ (Table 5): comprising Fe(III) pyrophosphate, sunflower lecithin, gum arabic (100% w/w), and pre-gelatinised rice starch (embodiment according to the invention, sucrester absent).

Note: “% w/w” means percentage by weight with respect to the total weight of polysaccharide (carrageenan or gum arabic) and sucrester.
Sideral RM V₀₀₀-M: comprising Fe(III) pyrophosphate, sunflower lecithin, sucrester and pre-gelatinised rice starch; does not include carrageenan, acacia gum and fucoidan (embodiment not according to the invention).

For each composition under analysis at least three tests were carried out on different animals in duplicate.

I.3. Results

Studies on isolated rat intestine allow to evaluate the permeability of the intestinal epithelium to Fe³⁺.

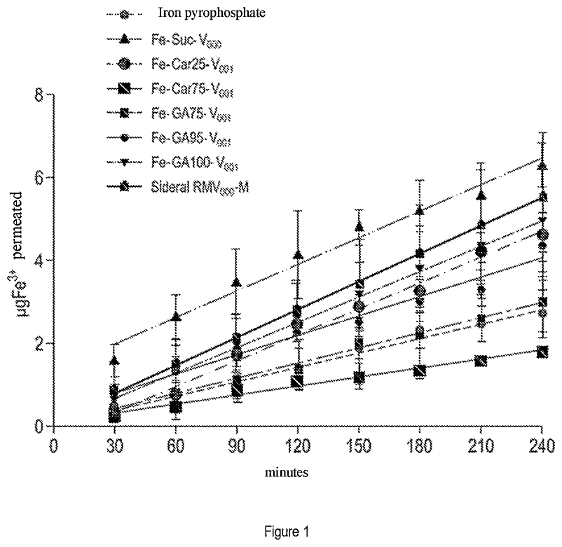

FIG. 1 shows the permeation data of Fe³⁺ through the isolated rat intestine. Since Fe³⁺ at the neutral pH of the intestine forms insoluble Fe(OH)₃, the ions of this type found in the receiving phase beyond the membrane can only have been transported as such encapsulated in nanosystems capable of being internalized by the intestinal epithelium cells.

From FIG. 1 and from table 8, in which the factors for enhancing the permeation of Fe³⁺ (enhancement ratio) are reported, we observe that the permeability of Fe³⁺ is effectively enhanced by: Fe-GA100V₀₀₁, Fe-Car25V₀₀₁ and, though to a lesser extent, from the composition Fe-GA95-V₀₀₁.

The formulations e-GA100V₀₀₁ and Fe-Car25V₀₀₁ are not significantly different from Fe-Suc-V₀₀₀ and from Sideral RM V₀₀₀-M.

Also the cumulative percentage permeated at the end of the experiment (4 h) in the case of the compositions Fe-GA100-V₀₀₁, Fe-Car25V₀₀₁ and Fe-GA95-V₀₀₁, is comparable with the cumulative percentage permeated in the case of the compositions Fe-Suc-V₀₀₀ and Sideral RM V₀₀₀-M.

In particular, in the case of carrageenan-based prototypes (Fe-Car25-V₀₀₁ and Fe-Car75V₀₀₁), it can be seen that by increasing the carrageenan percentage, the permeability of Fe³⁺ reduces.

Whereas the acacia gum compositions (Fe-GA25-V₀₀₁ and Fe-Car75V₀₀₁) behave in an opposite manner as compared to those based on carrageenan. As a matter of fact, by increasing the percentage of acacia gum, the permeability of Fe³⁺ increases.

These data indicate that acacia gum is capable of forming vesicles capable of transporting Fe³⁺ intact through the intestinal epithelium.

As reported above, Fe³⁺ at the neutral pH of the intestine forms Fe(OH)₃ which is substantially insoluble, and thus the ions of this type found in the receiving phase can only have been transported as such encapsulated in nanosystems capable of being internalized by the intestinal epithelium cells.

The compositions under analysis, in particular Sideral RM V₀₀₀-M, Fe-Car25-V₀₀₁ and Fe-GA100V₀₀₁, will also be tested on Caco-2, since the differences between the various compositions can be accentuated with this substrate to a greater extent than in the experiments in the isolated rat intestine model.

TABLE 8

| | Enhancement Ratio | |
|---|---|---|
| Formulation | (ER) | % permeate |
| Iron pyrophosphate | — | 0.21 ± 0.04 |
| Fe-Suc-V₀₀₀ | 2.12 | 0.47 ± 0.04 |
| Fe-Car25- V₀₀₁ | 2.07 | 0.34 ± 0.03 |
| Fe-Car75- V₀₀₁ | 0.70 | 0.14 ± 0.01 |
| Fe-GA75-V₀₀₁ | 1.14 | 0.22 ± 0.05 |
| Fe-GA95-V₀₀₁ | 1.47 | 0.33 ± 0.06 |
| Fe-GA100-V₀₀₁ | 1.95 | 0.37 ± 0.10 |
| Sideral RM V₀₀₀-M | 2.14 | 0.42 ± 0.11 |

II. Study of the Kinetics of Release of Fe³⁺ from the Compositions According to the Invention or Comparative Compositions

II.1 Method

The apparatus used for the determination of the kinetics of release of Fe³⁺ from the compositions under analysis consisted of an external circulation thermostat, regulated at a temperature of 37° C., and a beaker (internal diameter, 95 mm; height, 100 mm) provided with a thermostating jacket, placed on a lift, a synchronous motor at 120 rpm, which drove a paddle stirrer (length 49 mm, height 15 mm), into which 500 mg of the compositions to be tested were introduced.

At time t=0, the stirrer was submerged into the receiving phase (100 ml), contained in the beaker, and pre-thermostated to 37° C. The described apparatus allowed to control the hydrodynamics around the matrix.

A measured volume (1 ml) of the receiving phase, which was analysed under UV for the concentration of Fe³⁺ after centrifugation at 13400 rpm for 5 minutes, was collected at 30-minute intervals.

Unless indicated otherwise, the elution means were simulated gastrointestinal fluids, consisting of the following solutions:
Simulated gastric fluid (SGF), consisting of 0.04 M HCI, pH 1.2, made isotonic with NaCl (40 g of 1N HCI and 1 g of NaCl per 500 ml).

II.2. Composition Under Analysis

The compositions under analysis all contained the same iron concentration (1.34 mg/mL):
Fe-Suc-V₀₀₀: comprising Fe(III) pyrophosphate, sunflower lecithin, sucrester and pre-gelatinised rice starch; does not include carrageenan, acacia gum and fucoidan (embodiment not according to the invention, reference composition).
Fe-Car25-V₀₀₁ (Table 1): comprising Fe(III) pyrophosphate, sunflower lecithin, carrageenan (25% w/w) and sucrester (75% w/w), and pre-gelatinised rice starch (embodiment according to the invention).
Fe-Car75-V₀₀₁ (Table 2): comprising Fe(III) pyrophosphate, sunflower lecithin, carrageenan (75% w/w) and sucrester (25% w/w), and pre-gelatinised rice starch (embodiment according to the invention).
Fe-GA25-V₀₀₁ (Table 3): comprising Fe(III) pyrophosphate, sunflower lecithin, gum arabic (25% w/w) and sucrester (75% w/w), and pre-gelatinised rice starch (embodiment according to the invention).
Fe-GA75-V₀₀₁ (Table 4): comprising Fe(III) pyrophosphate, sunflower lecithin, gum arabic (75% w/w) and sucrester (25% w/w), and pre-gelatinised rice starch (embodiment according to the invention).
Fe-GA100-V₀₀₁ (Table 5): comprising Fe(III) pyrophosphate, sunflower lecithin, gum arabic (100% w/w), and pre-gelatinised rice starch (embodiment according to the invention, does not comprise sucrester).
Fe-FU25-V₀₀₁ (Table 6): comprising Fe(III) pyrophosphate, sunflower lecithin, fucoidan (25% w/w) and sucrester (75% w/w) and pre-gelatinised rice starch (embodiment according to the invention).
Fe-FU75-V₀₀₁ (Table 7): comprising Fe(III) pyrophosphate, sunflower lecithin, fucoidan (75% w/w) and sucrester (25% w/w), and pre-gelatinised rice starch (embodiment according to the invention).

For each composition under analysis at least three tests were carried out on in duplicate.

II.3. Results

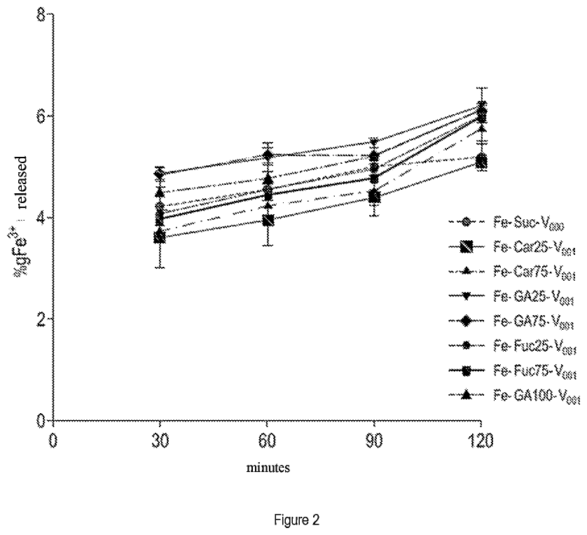

FIG. 2 shows the release profile of Fe³⁺ in a simulated gastric environment (pH 1,2) in the 2 hour interval of the compositions according to the invention based on carrageenan (Fe-Car25-V₀₀₁, Fe-Car75-V₀₀₁), acacia gum (Fe-GA25-V₀₀₁, Fe-GA75-V₀₀₁, Fe-GA100-V₀₀₁) and fucoidan (Fe-FU25-V₀₀₁, Fe-FU75-V₀₀₁) and of the reference composition (not according to the invention) Fe-Suc-V₀₀₀.

The data reported in FIG. 2 show that the compositions are able to control the release of Fe³⁺ into the stomach. Such release tests are a measure of the ability of the compositions according to the invention to encapsulate iron.

Therefore, should the compositions of the invention give rise to the extemporary formation of vesicles or vesicular structures, the iron remains encapsulated or bound to said vesicular structures.

III. Determination of the Presence of Particles Smaller Than a Micron in the Compositions Under Analysis and Determination of Size of the Micelles.

III. Method

1 g of each composition under analysis was dispersed in water. The dispersion was centrifuged at 2000 rpm for 10 minutes. Particles with dimensions above the micron, which sediment, can be separated from particles of smaller dimensions at this speed. The presence of a powder residue was observed in both cases. The supernatant, which was subjected to light scattering analysis (Coulter N4 plus), was collected.

III.2. Composition Under Analysis: Similar to Paragraph II.2.

III. Results

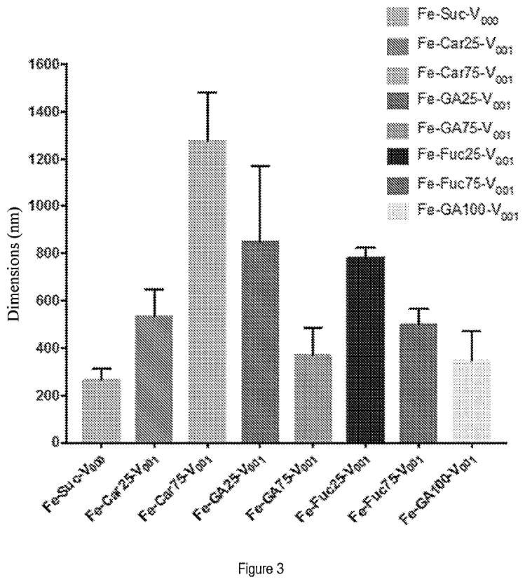

FIG. 3 shows the results of the light scattering analysis of the compositions according to the invention based on carrageenan (Fe-Car25-V₀₀₁, Fe-Car75-V₀₀₁), acacia gum (Fe-GA25-V₀₀₁, Fe-GA75-V₀₀₁, Fe-GA100-V₀₀₁) and fucoidan (Fe-FU25-V₀₀₁, Fe-FU75-V₀₀₁) and of the reference composition (not according to the invention) Fe-Suc-V₀₀₀

The data of FIG. 3 show that in the case of the compositions based on carrageenan according to the invention (Fe-Car25-V₀₀₁, Fe-Car75-V₀₀₁), the average size of the vesicles increases significantly as the presence of carrageenan increases.

It can be assumed that this proportional relationship is due to the fact that the polymer (carrageenan) is adsorbed on the surface of the vesicles.

In the case of the compositions based on acacia gum according to the invention (Fe-GA25-V₀₀₁, Fe-GA75-V₀₀₁, Fe-GA100-V₀₀₁) and fucoidan according to the invention (Fe-FU25-V₀₀₁, Fe-FU75-V₀₀₁), the data of FIG. 3 show that average vesicle dimensions decrease as the presence of both fucoidan and acacia gum increases.

It can be assumed that this inversely proportional relationship is due to the fact that both fucoidan and acacia gum act as emulsifier and stabiliser.

As a matter of fact, with the composition according to the invention Fe-GA100-V₀₀₁, the dimensions of the vesicles are not significantly different from the reference composition (Fe-Suc-V₀₀₀).

However, the polydispersity index (range 0.8-1.5, not reported) indicates that all the compositions according to the invention do not have homogeneous dimensions with respect to the mean and, in any case, they are not significantly different from the reference composition Fe-Suc-V₀₀₀.

TABLE 1

| | Amt (g) | Amt (mg) |
|---|---|---|
| Fe-Car25-V₀₀₁ | per 100 g | per unit |
| Jetomized iron pyrophosphate | 44.760 | 447.600 |
| Pre-gelatinised rice starch (Oryza sativa L.) | 37.520 | 375.200 |
| Carrageenan type CSW-2 | 4.285 | 42.85 |
| Sucrose fatty acid esters (sucrester) | 12.855 | 128.550 |
| Sunflower Lecithin | 0.580 | 5.800 |
| Total | 100.00 | 1000.000 |

TABLE 2

| | Amt (g) | Amt (mg) |
|---|---|---|
| Fe-Car75-V₀₀₁ | per 100 g | per unit |
| Jetomized iron pyrophosphate | 44.760 | 447.600 |
| Pre-gelatinised rice starch (Oryza sativa L.) | 37.520 | 375.200 |
| Carrageenan type CSW-2 | 12.855 | 42.85 |
| Sucrose fatty acid esters (sucrester) | 4.285 | 128.550 |
| Sunflower Lecithin | 0.580 | 5.800 |
| Total | 100.00 | 1000.000 |

TABLE 3

| | Amt (g) | Amt (mg) |
|---|---|---|
| Fe-GA25-V₀₀₁ | per 100 g | per unit |
| Jetomized iron pyrophosphate | 44.760 | 447.600 |
| Pre-gelatinised rice starch (Oryza sativa L.) | 37.520 | 375.200 |
| Acacia gum | 4.285 | 42.85 |
| Sucrose fatty acid esters (sucrester) | 12.855 | 128.550 |
| Sunflower Lecithin | 0.580 | 5.800 |
| Total | 100.00 | 1000.000 |

TABLE 4

| | Amt (g) | Amt (mg) |
|---|---|---|
| Fe-GA75-V₀₀₁ | per 100 g | per unit |
| Jetomized iron pyrophosphate | 44.760 | 447.600 |
| Pre-gelatinised rice starch (Oryza sativa L.) | 37.520 | 375.200 |
| Acacia gum | 12.855 | 42.85 |
| Sucrose fatty acid esters (sucrester) | 4.285 | 128.550 |
| Sunflower Lecithin | 0.580 | 5.800 |
| Total | 100.00 | 1000.000 |

TABLE 5

| | Amt (g) | Amt (mg) |
|---|---|---|
| Fe-GA100-V₀₀₁ | per 100 g | per unit |
| Jetomized iron pyrophosphate | 44.760 | 447.600 |
| Pre-gelatinised rice starch (Oryza sativa L.) | 37.520 | 375.200 |
| Acacia gum | 17.140 | 42.85 |
| Sunflower Lecithin | 0.580 | 5.800 |
| Total | 100.00 | 1000.000 |

TABLE 6

| | Amt (g) | Amt (mg) |
|---|---|---|
| Fe-Fu25-V₀₀₁ | per 100 g | per unit |
| Jetomized iron pyrophosphate | 44.760 | 447.600 |
| Pre-gelatinised rice starch (Oryza sativa L.) | 37.520 | 375.200 |
| Fucoidan 85% | 4.285 | 42.85 |
| Sucrose fatty acid esters (sucrester) | 12.855 | 128.550 |
| Sunflower Lecithin | 0.580 | 5.800 |
| Total | 100.00 | 1000.000 |

TABLE 7

| | Amt (g) | Amt (mg) |
|---|---|---|
| Fe-Fu75-V₀₀₁ | per 100 g | per unit |
| Jetomized iron pyrophosphate | 44.760 | 447.600 |
| Pre-gelatinised rice starch (Oryza sativa L.) | 37.520 | 375.200 |
| Fucoidan 85% | 12.855 | 42.85 |
| Sucrose fatty acid esters (sucrester) | 4.285 | 128.550 |
| Sunflower Lecithin | 0.580 | 5.800 |
| Total | 100.00 | 1000.000 |

1. A formulation in solid form comprising:
a nutrient, wherein said nutrient is a mineral or a vitamin,
wherein said mineral is selected from the group consisting of: magnesium, calcium, iron, zinc, iodine, selenium, chromium, and copper (II), and wherein said mineral is in the form of a salt, complex or oxide of said mineral; and
wherein said vitamin is selected from the group consisting of: vitamin B12, vitamin C, vitamin D3, and vitamin E;
a phospholipid; and
a first agent selected from a carrageenan or an acacia gum.

2. The formulation according to claim 1, wherein said formulation further comprises at least one sucrester or a fatty acid carbohydrate ester, the at least one sucrester or a fatty acid carbohydrate ester comprising from 70% to 90% by weight, with respect to the total weight of the sucrester, of monoesters obtained through the esterification of sucrose with one or more fatty acids of plant origin.

3. The formulation according to claim 1, wherein said first agent consists of said carrageenan.

4. The formulation according to claim 1, wherein said first agent consists of said acacia gum.

5. The formulation according to claim 1, wherein said formulation further comprises a gelatinised or pre-gelatinised starch of plant origin.

6. The formulation according to claim 1, wherein said phospholipid is a phosphatidylcholine or lecithin.

7. The formulation according to claim 1, wherein a weight ratio between said first agent and said phospholipid.

8. The formulation according to claim 2, wherein on 100 parts by weight of a sum of said first agent and said sucrester, said first agent is comprised in percentage by weight from 1% to 100% and said sucrester is comprised from 0% to 99%.

9. A composition comprising:
at least one solid form formulation of said nutrient according to claim 1; and
at least one acceptable pharmaceutical or food grade additive and/or excipient.

10. The composition according to claim 9, wherein said nutrient is a mineral.

11. The composition according to claim 9, wherein said nutrient is a vitamin.

12. The composition according to claim 9, wherein said composition comprises:
at least one solid form formulation of the mineral;
at least one solid form formulation of the vitamin; and at least one acceptable pharmaceutical or food grade additive and/or excipient.

13. A method to treat a subject, the method comprising administering to the subject the formulation according to claim 1 or a composition including the said formulation, as a medicament.

14. A method for the preventive, curative and/or adjuvant treatment of a deficiency of said mineral and/or vitamin, and of diseases, symptoms or disorders related to or deriving from said deficiency, the method comprising
administering to a subject in need a therapeutically effective amount of the formulation according to claim 1 or a composition comprising said formulation.

15. The method according to claim 14, wherein said diseases or symptoms related to or deriving from said deficiency are selected from the group consisting of:
sideropenia, anaemia, poor iron absorption, haemolytic anaemia;
changes in carbohydrate metabolism and related diseases and disorders, diabetes, type II diabetes mellitus, hyperglycaemia, insulin resistance, high absorption of carbohydrates, deregulation of blood glucose level, metabolic syndrome;
changes in the muscle energy metabolism and/or disorders related thereto decrease in muscle mass, decrease in muscle strength, decrease in physical resistance to muscle stress, poor absorption of amino acids;
dyslipidaemia or change in the lipid metabolism and diseases and disorders related thereto, cholesterolaemia, high triglyceride levels, and obesity or overweight;
cognitive disorders or changes in the cognitive-emotional sphere;
cardiometabolic disorders;
change in the immune system;
stress related to anxiety and depression, fatigue, chronic fatigue, asthenia, a lack of reflexes and coordination;
rickets or delayed bone growth in children, osteomalacia in adults, osteoporosis, and hyperparathyroidism;
gingivitis; and
brittleness of the nails, hair and/or skin following inflammations or medical treatment.

16. A method for supplementing a mineral and/or vitamin, the method comprising
administering to a healthy subject an effective amount of the formulation according to claim 1 or a composition comprising said formulation for the supplementation of said mineral and/or vitamin in the healthy subject.

17. The method according to claim 16, wherein said the supplementation is performed for increasing muscle mass, increasing muscle strength, increasing physical resistance to muscle stress, reducing strength recovery time after a physical effort, increasing mental performance, strengthening nails and hair.

18. A process for the preparation of the formulation according to claim 1 comprising:
providing a mineral or a vitamin in the form of powder or granules, wherein said mineral is in the form of a salt, oxide or complex of said mineral;
mixing, in the absence of solvent, said mineral or said vitamin with a phospholipid to obtain a mineral or vitamin-phosphoplipid mixture;
mixing, in the absence of solvent, said mineral or vitamin-phosphoplipid mixture with a polysaccharide selected from carrageenan or acacia gum and, optionally, with a sucrester, to obtain the formulation.

19. A process for the preparation of the composition according to claim 12, the method comprising:
providing at least one solid form formulation of a mineral and at least one solid form formulation of a vitamin
wherein said mineral is selected from the group consisting of: magnesium, calcium, iron, zinc, iodine, selenium, chromium, and copper (II), and wherein said mineral is in the form of a salt, complex or oxide of said mineral; and
wherein said vitamin is selected from the group consisting of: vitamin B12, vitamin C, vitamin D3, and vitamin E;
mixing said at least one solid form formulation of a mineral and said at least one solid form formulation of a vitamin to obtain a mineral-vitamin mixture;
mixing said mineral-vitamin mixture with at least one additive and/or excipient to obtain the composition.

20. A process for the preparation of the formulation according to claim 1 comprising:
providing a mineral or a vitamin in the form of powder or granules, wherein said mineral is in the form of a salt, oxide or complex of said mineral;
mixing, in the absence of solvent, said mineral or said vitamin with a phospholipid to obtain a mineral or vitamin-phosphoplipid mixture;
mixing, in the absence of solvent, said mineral or vitamin phosphoplipid mixture with a polysaccharide selected from carrageenan or acacia gum and, optionally, with a sucrester, to obtain a mineral or vitamin-phosphoplipid-polysaccharide mixture; and
mixing said another mineral or vitamin-phosphoplipid-polysaccharide mixture with a gelatinised or pre-gelatinised starch of plant origin, to obtain the formulation.
